(19) United States
(12) Patent Application Publication
(10) Pub. No.: US 2016/0082037 A1
(43) Pub. Date: Mar. 24, 2016

(54) COMPOSITION, PREPARATION, AND USE OF CHITOSAN POWDER FOR BIOMEDICAL APPLICATIONS

(71) Applicants: LOMA LINDA UNIVERSITY MEDICAL CENTER, Loma Linda, CA (US); North Carolina State University, Raleigh, NC (US); LOMA LINDA UNIVERSITY, Loma Linda, CA (US)

(72) Inventors: Wolff M. Kirsch, Redlands, CA (US); Samuel M. Hudson, Raleigh, NC (US); Andrew Crofton, Loma Linda, CA (US)

(21) Appl. No.: 14/863,270

(22) Filed: Sep. 23, 2015

Related U.S. Application Data
(60) Provisional application No. 62/054,343, filed on Sep. 23, 2014.

(51) Int. Cl. A61K 31/722; A61L 2/14; A61L 2/08

(52) U.S. Cl. CPC A61K 31/722; A61L 2/081; A61L 2/14

(57) ABSTRACT

A powder chitosan-based material can be used for biomedical applications. The chitosan has been treated in a nitrogen field by applying energy to ionize nitrogen in and around the chitosan material. A single or multiple such treatments may be employed. For example, the chitosan material may be irradiated under nitrogen using γ-irradiation, treated under a nitrogen plasma, or both. A powder chitosan material can be readily treated by surface modifying treatments such as irradiating under nitrogen using γ-irradiation, treating under a nitrogen plasma, or both.

### INCORPORATION BY REFERENCE TO RELATED APPLICATION

Any and all priority claims identified in the Application Data Sheet, or any correction thereto, are hereby incorporated by reference under 37 CFR 1.57. This application claims the benefit of U.S. Provisional Application No. 62/054,343 filed Sep. 23, 2014. The aforementioned application is incorporated by reference herein in its entirety, and is hereby expressly made a part of this specification.

### BACKGROUND

1. Field of the Invention

Hemostatic materials made from chitosan are provided, more particularly, chitosan shards having reduced pyrogenicity.

2. Description of the Related Art

Surgical procedures and traumatic injuries are often characterized by massive blood loss. Conventional approaches such as manual pressure, cauterization, or sutures may be time consuming and are not always effective in controlling bleeding.

Over the years, a number of topical hemostatic agents have been developed to control bleeding during surgical procedures and to control bleeding resulting from traumatic injury. Some agents such as collagen-based powders, sponges, or cloths are of a particulate nature. Particulate hemostatic agents provide a lattice for natural thrombus formation, but are unable to enhance this process in coagulopathic patients. Microfibrillar collagen, a particulate hemostatic agent, comes in powder form and stimulates the patient's intrinsic hemostatic cascade. However, this product has been reported to embolize and induce a localized inflammatory response if used during cardiopulmonary bypass. Further, particulates such as powders and even gels are difficult to control, and are easily carried away from an active bleeding site.

Pharmacologically-active agents such as thrombin can be used in combination with a particulate carrier, for example, as in a gelfoam sponge or powder soaked in thrombin. Thrombin has been used to control bleeding on diffusely bleeding surfaces, but the lack of a framework onto which the clot can adhere has limited its use. The autologous and allogenic fibrin glues can cause clot formation, but do not adhere well to wet tissue and have little impact on actively bleeding wounds.

Chitosan, the N-deacetylated derivation of chitin, has demonstrated hemostatic effectiveness as well as biocompatibility, biodegradability, and anti-bacterial activity. Chitosan has been shown to secure mucoadhesion and hemostasis despite defibrination and anticoagulation. FDA approved topical chitosan hemostats include Celox™ (a granular powder) and HemCon (a lyophilized chitosan film). Also FDA approved, for external use, is a microfibrillar high molecular weight chitosan in the form of sponge, puff or non-woven fabric.

Although chitosan has been shown to be an effective hemostat, the traditional, inexpensive methods for manufacturing commodity-grade chitosan yields a product that is laden with pyrogens, particularly endotoxins, which limit its applicability in the biological and medical arenas, as minute amounts of endotoxins may induce septic responses when contacted with mammalian tissue.

Experimental and biocompatibility artifacts are generated by unappreciated endotoxins in commercially available, “medical grade” chitosan. For example, an “ultra-pure” chitosan (PROTASAN™ S-213 frp, NovaMatrix™, a business unit of FMC BioPolymer, Sandvika, Norway) was advertised as having an endotoxin burden<100 EU/gm, a level that prohibits implantation but allows topical applications. However, an independent analysis of Protasan S-213 found endotoxin levels in the hemostat to be 247 EU/gm, over two times higher than the manufacturer's guaranteed level. Additionally, a medical-grade chitosan marketed as POLYPROLATE™ derived from Dungeness crab shells (Scion Cardio-Vascular, Inc., Miami, Fla.) has an EU level at 28 EU/gm. The FDA requires an EU level below 20 EU/gm for implantable medical devices which includes chitosan hemostats. The lack of an FDA-approved, implantable depyrogenated chitosan has halted advancements and development of promising techniques utilizing internal or implantable chitosan materials.

### SUMMARY

There is a need for a chitosan-based hemostatic material having reduced levels of pyrogens, and/or one in which the endotoxins have been removed and/or inactivated sufficiently to avoid inducing septic responses when contacted with mammalian tissue. Additionally, the ability to produce chitosan substances that are more effectively depyrogenated or purified is desirable.

In accordance with a first aspect, a method is provided of making a material comprising: processing chitosan into a powder chitosan material by a physical process; irradiating the powder chitosan material under nitrogen plasma; and combining the powder chitosan material with an acid to create a viscous solution.

In an embodiment of the first aspect, the method further comprises utilizing chitosan flakes directly obtained from shellfish, the chitosan flakes having an average largest dimension of 1 centimeter or more.

In an embodiment of the first aspect, the method further comprises grinding the chitosan flakes with zirconium grinding balls.

In an embodiment of the first aspect, the physical process comprises grinding of the chitosan to form the powder chitosan material.

In an embodiment of the first aspect, an individual granule of the powder chitosan material is 10 μm to 100 μm in diameter.

In an embodiment of the first aspect, an average diameter of individual granules of the powder chitosan material is from 10 μm to 100 μm in diameter.

In an embodiment of the first aspect, the acid is selected from the group consisting of acetic acid, lactic acid, glutamic acid, and formic acid.

In an embodiment of the first aspect, the acid is lactic acid.

In an embodiment of the first aspect, the method further comprises processing the viscous solution into a hemostatic device comprising a network of the powder chitosan material.

In an embodiment of the first aspect, the method further comprises processing the viscous solution into a drug delivery device comprising a network of the powder chitosan material and a drug.

In an embodiment of the first aspect, the method further comprises treating the powder chitosan material under a nitrogen plasma.

In an embodiment of the first aspect, the method further comprises soaking the powder chitosan material in an alcohol prior to treating with γ-irradiation or plasma.

In an embodiment of the first aspect, the method further comprises treating the powder chitosan material under the nitrogen plasma for 30 minutes or more.

In a second aspect, a method is provided of making a material comprising: processing chitosan into a powder chitosan material by a physical process; and irradiating the powder chitosan material under a nitrogen plasma while rotating the powder chitosan material during nitrogen plasma irradiation.

In an embodiment of the second aspect, the rotating is conducted by a tumbler plasma chamber.

In an embodiment of the second aspect, the method further comprises utilizing chitosan flakes directly obtained from shellfish, the chitosan flakes having an average largest dimension of 1 centimeter or more.

In an embodiment of the second aspect, the method further comprises grinding the chitosan flakes with zirconium grinding balls.

In an embodiment of the second aspect, the physical process comprises grinding of the chitosan to form the powder chitosan material.

In an embodiment of the second aspect, an individual granule of the powder chitosan material is 10 μm to 100 μm in diameter.

In an embodiment of the second aspect, an average diameter of individual granules of the powder chitosan material is from 10 μm to 100 μm in diameter.

In an embodiment of the second aspect, the method further comprises treating the powder chitosan material under a nitrogen plasma.

In an embodiment of the second aspect, the method further comprises soaking the powder chitosan material in an alcohol prior to treating with γ-irradiation or plasma.

In an embodiment of the second aspect, the method further comprises additionally comprising treating the powder chitosan material under the nitrogen plasma for 30 minutes.

In a third aspect, an apparatus is provided comprising: a drug delivery device, the device comprising a powder material, the powder material comprising chitosan, wherein the powder material is γ-irradiated under nitrogen.

In an embodiment of the third aspect, the powder material is configured to be processed into a hemostatic device comprising a network of powder material dissolved in an acid.

In an embodiment of the third aspect, the powder material is configured to be processed into a drug delivery device comprising a network of the powder material dissolved in an acid.

In an embodiment of the third aspect, the powder material is formed from a physical process comprises grinding of chitosan flakes to form the powder material.

In an embodiment of the third aspect, an individual granule of the powder material is 10 μm to 100 μm in diameter.

In an embodiment of the third aspect, the powder material is treated under a nitrogen plasma.

In an embodiment of the third aspect, the powder material is soaked in an alcohol prior to treating with γ-irradiation or plasma.

In an embodiment of the third aspect, the powder material is treated under the nitrogen plasma for 30 minutes or more.

In an embodiment of the third aspect, the apparatus contains <20 endotoxin units.

In an embodiment of the third aspect, <20 endotoxin units per gram of chitosan are present.

### BRIEF DESCRIPTION OF THE DRAWINGS

FIG. 1 schematically depicts a process for obtaining chitosan from crustacean shell waste in accordance with one embodiment.

FIG. 1B schematically depicts an embodiment of an apparatus for preparing chitosan fibers.

FIG. 1C provides a schematic of an assembly line for production of chitosan fleece in accordance with one embodiment.

FIG. 2 illustrates a schematic of one embodiment of a North Carolina Atmospheric Plasma System (NCAPS).

FIG. 3 is a schematic depiction of one embodiment of a plasma treatment assembly.

FIGS. 4A-D illustrate embodiments of commercially available “medical-grade” forms of chitosan.

FIGS. 5A-D illustrate Scanning Electron Microscopy (SEM) comparisons of the surface areas for microfibrillar chitosan and a HemCon® lyophilized dressing.

FIG. 6A illustrates embodiments of solution processed chitosan fibrids, including Materials “A”, “B”, “C”, “D”, and “E”.

FIG. 6B illustrates an embodiment of a conventional wet spun chitosan filament.

FIG. 7 illustrates an SEM image of an embodiment of chitosan fibrids that are directly obtained by shear induced precipitation of chitosan from solution as shown in Material “A” of FIG. 6A.

FIGS. 8A-B illustrates SEM images of individual chitosan fibrid pulp obtained by the direct precipitation of sheared chitosan solutions.

FIG. 9A illustrates an SEM image of chitosan fibrids processed with methanol and obtained from shear precipitated chitosan solution.

FIG. 9B illustrates an SEM image of a film or paper-like structure obtained from chitosan fibrids.

FIG. 10 is an embodiment of a chitosan pad.

FIGS. 11A-C show pictures of various small particle chitosan materials.

FIG. 12 is a picture of chitosan flakes that can be treated with depyrogenation techniques.

FIGS. 13A-B are pictures of chitosan powder, chitosan flakes, and chitosan pads.

FIG. 14 shows the results from cryomilled chitosan material.

FIGS. 15A-B show chitosan powders that have been cryomilled.

FIGS. 16A-E illustrate scanning electron microscopic images of chitosan flakes before and after milling.

### DETAILED DESCRIPTION

Chitosan is obtained from chitin, a widely available biopolymer obtained principally from shrimp and crab shell waste. Chitosan is the main derivative of chitin, and is the collective term applied to deacetylated chitins in various stages of deacetylation and depolymerization. The chemical structure of chitin and chitosan is similar to that of cellulose. The difference is that instead of the hydroxyl group as is bonded at C-2 in each D-glucose unit of cellulose, there is an acetylated amino group (—NHCOCH₃) at C-2 in each D-glucose unit in chitin and an amino group at C-2 in each D-glucose unit of chitosan.

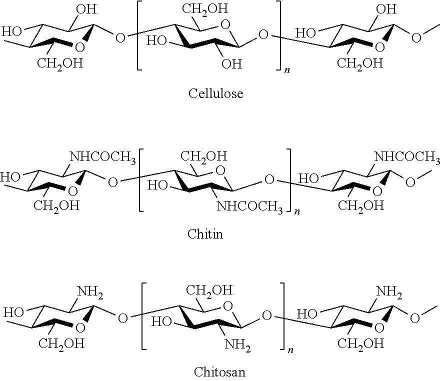

Chitin and chitosan are both nontoxic, but chitosan is used more widely in medical and pharmaceutical applications than chitin because of its good solubility in acid solution. Chitosan has good biocompatibility and is biodegradable by chitosanase, papain, cellulase, and acid protease. Chitosan exhibits anti-inflammatory and analgesic effects, and promotes hemostasis and wound healing. Chitosan has also been shown to be an effective hemostatic agent. Chitosan hemostasis is believed to be mediated by positively charged amine groups binding to negatively charged red cell and platelet surfaces forming a mucoadhesive coagulum without activation of classical coagulation pathways.

In an embodiment, a hemostatic device made from chitosan can be constructed in the form of sponge, puff or non-woven fabric. The chitosan materials are discussed in U.S. Publ. No. 2005/0123588 A1 and U.S. Publ. No. 2005/0240137 A1. The entirety of both of these published patent applications, and particularly the disclosure directed to making and using chitosan-based hemostatic devices, is hereby incorporated by reference in its entirety. Other forms of chitosan known in the art can also be used as described herein.

Additionally, chitosan can be an effective drug delivery vehicle due to the anti-inflammatory and analgesic effects. Chitosan can also be an effective drug delivery vehicle due to its ability to adhere to tissues, loosen gap junctions, and incorporate therapeutic compounds under mild conditions. The use of chitosan nanoparticles as a drug delivery device to deliver inhibitors (alphaGal lectin, anti-C5 Mab, C1-Inhibitor, factor H, human CD59 cDNA) to the brain for treatment of cerebral amyloid angiopathy is discussed in Applicants' copending U.S. application Ser. No. 14/677,953, filed Apr. 2, 2015, and directed to “SUBSTANCES AND METHODS FOR THE TREATMENT OF CEREBRAL AMYLOID ANGIOPATHY RELATED CONDITIONS OR DISEASES” and International Patent Application No. PCT/US2013/030582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of these applications is hereby incorporated by reference.

As discussed above, chitosan is formed from chitin, which is present in crustacean shells as a composite with proteins and calcium salts. Chitin is produced by removing calcium carbonate and protein from these shells, and chitosan is produced by deacetylation of chitin in a strong alkali solution.

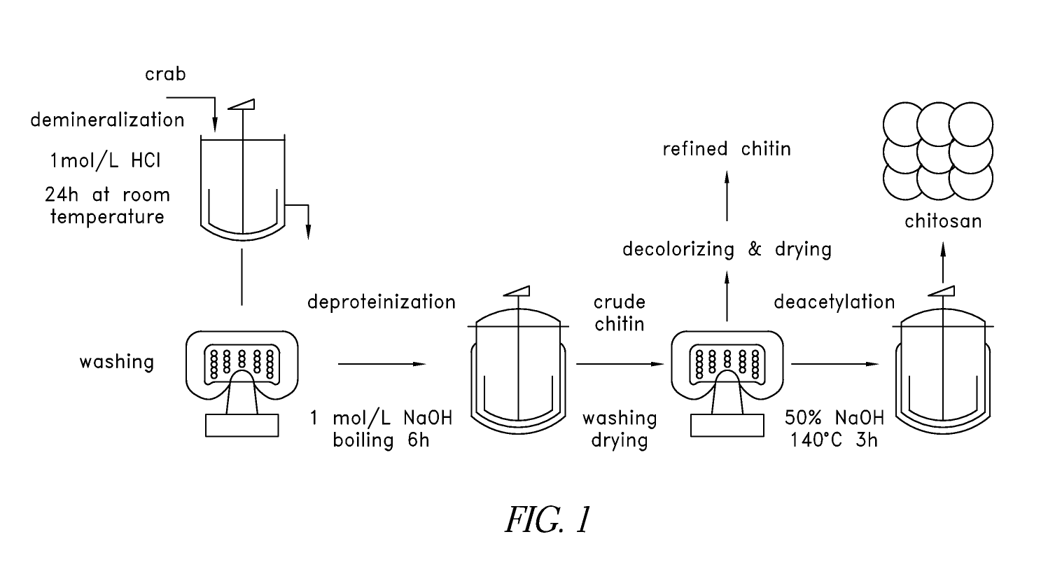

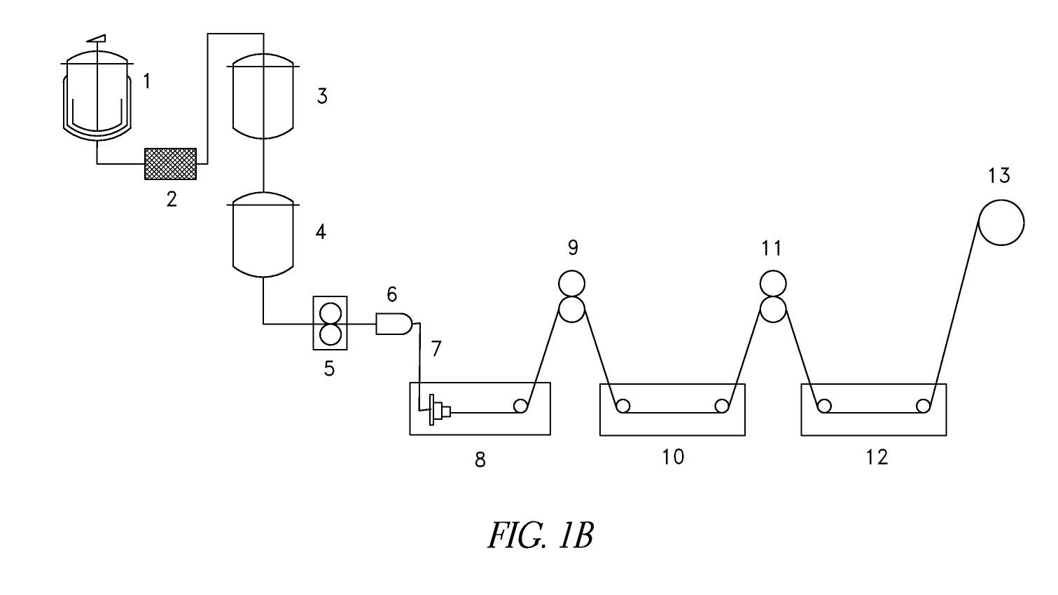

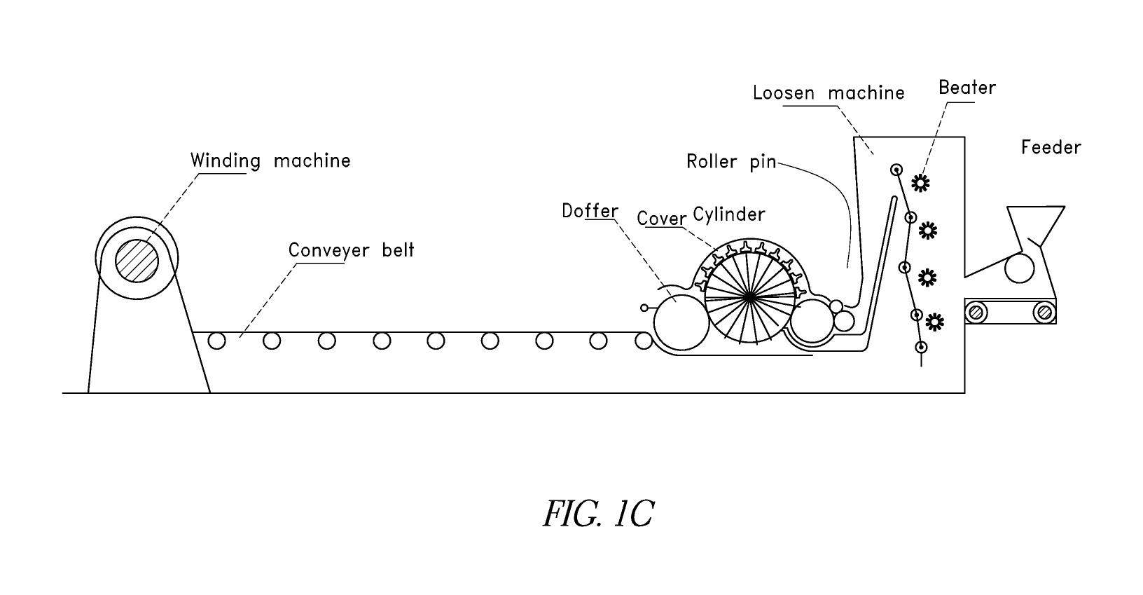

One method for obtaining chitosan from crab, shrimp or other crustacean shells is schematically depicted in FIG. 1 and described as follows. Calcium carbonate is removed by immersing the shell in dilute hydrochloric acid at room temperature for 24 hours (demineralization). Proteins are then extracted from the decalcified shells by boiling them with dilute aqueous sodium hydroxide for six hours (deproteinization). The demineralization and deproteinization steps can be repeated at least two times to remove substantially all of the inorganic materials and proteins from the crustacean shells. The crude chitin thus obtained is washed then dried. The chitin is heated at 140° C. in a strong alkali solution (50 wt. %) for 3 hours. Highly deacetylated chitosan exhibiting no significant degradation of molecular chain is then obtained by intermittently washing the intermediate product in water two or more times during the alkali treatment.

Chitosan fibers can be prepared by a wet spinning method, although any suitable method could be used. In other embodiments, the chitosan can be prepared into thin films and/or chitosan shards through compression, shredding, slitting, and/or other techniques as is described in detail in U.S. application Ser. No. 14/254,827, filed Apr. 16, 2014 entitled “COMPOSITION, PREPARATION, AND USE OF CHITOSAN SHARDS FOR BIOMEDICAL APPLICATIONS” The entirety of this application is hereby incorporated by reference. In one embodiment, chitosan is first dissolved in a suitable solvent to yield a primary spinning solution. Solvents can include acidic solutions, for example, solutions containing trichloroacetic acetic acid, acetic acid, lactic acid, and the like, however any suitable solvent can be employed. The primary spinning solution is filtered and deaerated, after which it is sprayed under pressure into a solidifying bath through the pores of a spinning jet. Solid chitosan fibers are recovered from the solidified bath. The fibers can be subjected to further processing steps, including but not limited to drawing, washing, drying, post treatment, functionalization, and the like.

FIG. 1B illustrates an apparatus for preparing chitosan fibers in accordance with one embodiment. The illustrated apparatus includes a dissolving kettle 1, a filter 2, a middle tank 3, a storage tank 4, a dosage pump 5, a filter 6, a spinning jet 7, a solidifying bath 8, a pickup roll 9, a draw bath 10, a draw roll 11, a washing bath 12, and a coiling roll 13.

In one embodiment, the primary chitosan spinning solution is prepared by dissolving 3 parts chitosan powder in a mixed solvent at 5° C. containing 50 parts trichloroacetic acid (TDA) to 50 parts methylene dichloride. The resulting primary spinning solution is filtered and then deaerated under vacuum. A first solidifying bath comprising acetone at 14° C. is employed. The aperture of the spinning jet is 0.08 mm, the hole count is forty-eight, and the spinning velocity is 10 m/min. The spinning solution is maintained at 20° C. by heating with recycled hot water. The chitosan fibers from the acetone bath are recovered and conveyed via a conveyor belt to a second solidifying bath comprising methanol at 15° C. The fibers are maintained in the second solidifying bath for ten minutes. The fibers are recovered and then coiled at a velocity of 9 m/min. The coiled fibers are neutralized in a 0.3 g/l KOH solution for one hour, and are then washed with deionized water. The resulting chitosan fiber is then dried, after which it is ready for fabrication into the hemostatic materials of some embodiments.

In one embodiment, glacial, or anhydrous, acetic acid is employed as an agent to adhere the chitosan fibers to each other in embodiments where chitosan fibers, either alone or with an added medicament, therapeutic agent or other agent, are used in forming a hemostatic agent. In addition to providing good adherence between the chitosan fibers, fibers treated with glacial acetic acid also exhibit exceptional ability to adhere to wounds, including arterial or femoral wounds.

Depending upon the application, the concentration of acetic acid in solution can be adjusted to provide the desired degree of adhesion. For example, it can be desirable to employ a reduced concentration of acetic acid if the chitosan fibers are to be employed in treating a seeping wound or other wound where strong adhesion is not desired, or in applications where the hemostatic agent is to be removed from the wound. In such embodiments, an acetic concentration of from about 1 vol. % or less to about 20 vol. % is generally employed, and a concentration of from about 2, 3, 4, 5, 6, 7, 8, 9, or 10 vol. % to about 11, 12, 13, 14, 15, 16, 17, 18, or 19 vol. % is employed. Where strong adhesion between fibers, or strong adhesion to the wound is desired, a concentration can be greater than or equal to about 20 vol. %, from about 50, 55, 60, 65, or 70 vol. % to about 75, 80, 85, 90, 95, or 100 vol. %, or from about 95, 96, 97, 98, or 99 vol. % to about 100 vol. %.

Chitosan textile can be prepared from chitosan fibers using equipment commonly employed in the textile industry for fiber production. With reference next to FIG. 1C, an assembly line for production of chitosan fleece can employ a feeder, a loosen machine, a carding machine, a conveyor belt, and lastly a winding machine, as depicted below. In the feeder, chitosan short fiber is fed through a feeder and into a loosen machine, wherein chitosan short fiber is loosened by several beaters. In the carding machine, chitosan fibers are ripped and turned into chitosan fleece by high speed spinning of a cylinder and roller pin, then the fleece is peeled off as a separated thin layer of net by a duffer.

The production of fibers and associated processing discussed above is most effective when using chitosan of relatively high molecular weight. Such high molecular weight chitosan is particularly amenable to formation into fibrous forms such as fleece that can be formed into a strong and durable textile that is flexible and malleable but retains continuity so that it can be moved as a unit and doesn't break apart when manipulated during use. In some embodiments, chitosan fibers can be formed into a yarn, which in turn can be woven. In other embodiments, successive layers of chitosan fiber pieces can be flattened and sprayed with an acidic solution such as the glacial acetic acid discussed above so as to form a non-woven textile. In some examples, the solution has a pH of about 3.0-4.5.

In some embodiments, a fibrous hemostatic device is constructed of high molecular weight chitosan (e.g., greater than about 600 kDA). The high molecular weight chitosan lends itself to construction of a dry, fibrous hemostatic material that can be constructed as a textile in a puff, fleece, fabric or sheet form. Embodiments of a chitosan-based hemostatic textile can be provided in many forms depending upon the nature of the wound and the treatment method employed. In some embodiments, a fibrous hemostatic device can be constructed of chitosan with standard molecular weight. Such chitosan devices, similar to high molecular weight chitosan, can be amenable to formation into fibrous forms of strong durable textiles that are flexible and malleable but retain continuity to be moved as a unit and not break apart when manipulated during use or for treatments as discussed herein. In other embodiments, chitosan of lower molecular weight (e.g., less than or equal to 600 kDa) can be employed.

Chitosan can then be formed into a chitosan powder, shards, films, ribbons, fibrids, fiber, sponges, flakes, slurries, microparticles, nanoparticulates, gels, solutions, aerosols, and/or other similar product that can be used to produce chitosan solutions, gels, foams, textiles, or pad. The chitosan solution, gel, foam, textile, and/or pad can be applied topically or implanted internally as a hemostatic agent and/or a drug delivery device. In some embodiments, the chitosan solution, gel, foam, textile, and/or pad can be a combination of any and/or all formations of the chitosan.

Normally, however, chitosan is laden with pyrogens, particularly endotoxins, which can limit its applicability in the biological and medical arenas, as minute amounts of endotoxins may induce septic responses when contacted with mammalian tissue. As such, in accordance with some embodiments, chitosan materials are used externally so as to minimize the likelihood of a septic response. In other embodiments, such chitosan materials can be used during surgeries, but only for temporary purposes, and are not implanted or left within a patient.

Endotoxins are essentially the skeletal or cellular remains and by-product secretions of dead bacteria, which are ubiquitous and found in the air, on surfaces and in food and water. More precisely, endotoxins are complex amphiphilic lipopolysaccharides (LPS) having both polysaccharide and lipophilic components. They are composed of pieces of the lipopolysaccharide wall component of Gram-negative bacteria. An example of LPS is shown below.

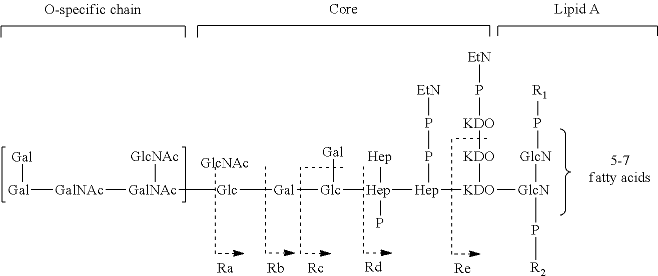

The terms endotoxin and pyrogen are often used interchangeably. Endotoxins are one of many pyrogens, which are substances that elicit a fever response in the bloodstream of a mammalian body. Vascular or lymphatic exposure to endotoxins can lead to severe sepsis, septic shock, and potential death. Thus, endotoxins are of particular concern to those manufacturing medical devices as they are one of the most potent pyrogens that can contaminate a product.

As such, pharmaceuticals, medical devices and products that contact human tissue, blood, bone or that can be absorbed by the body or implanted within the body must meet stringent levels of endotoxin control. The US Pharmacopeia has set specifications for endotoxin units (EU) for medical devices. The current standard (USP27) specifies <20 EU per device (e.g. <0.5 EU/mL in water). Some embodiments of chitosan-based hemostats anticipated for internal use have sufficiently reduced levels of endotoxins to comply with such standards. In the context of the embodiments, a “device” can include a unit dosage form of a liquid or solid pharmaceutical composition, e.g., a capsule, a tablet, a bolus, or the like, or multiple dosages intended to be administered sequentially or simultaneously, wherein an aggregate of the multiple dosages is a total dose to be administered to a patient in one treatment. Additionally, for example, a unit dosage of a pharmaceutical composition could require a maximum endotoxin load of <0.5 EU per gram for the chitosan-based pharmaceutical. In other examples, a “device” can include a medical device that can be implanted into the body and/or a body cavity for the treatment of a targeted area.

In some embodiments, multiple devices and/or multiple doses of the pharmaceutical can be administered at one time or within a certain timeframe. The cumulative endotoxin level for all devices cannot exceed the USP27 standard of <20 EU per device (e.g. <0.5 EU/mL in water) and/or <0.5 EU per gram. Therefore, in some embodiments, the endotoxin amount can influence the number of devices that can be implanted at one time. For example, 2 devices that contain 10 EU/device could be implanted in one surgery (20 EU total). For pharmaceuticals, since they are given at varying time intervals, the endotoxin quantity can influence the amount of drug that can be given in a certain window of time. Additionally, in some embodiments, the pharmaceutical composition with chitosan can be a 2% solution which will reduce the amount of endotoxins in the chitosan solution enabling a large dosage to be given in the event that endotoxin contamination levels are a concern.

Further, in some embodiments, the allowable endotoxin limits for internal uses of a device or pharmaceutical over time can be based on characteristics of the patient, for example the patient's height and/or weight. The endotoxin effect within the body is related to an immune system response to the presence of endotoxins. Therefore, once the endotoxin load is processed and/or expelled from the body and the body has substantially cleared the endotoxins from the previous implantation or administration additional devices and/or doses containing endotoxins can be introduced into the body. The endotoxins present in the patient due to the medical device or pharmaceutical can be expelled from the body through normal body processes. In some embodiments, the endotoxins can be processed and expelled within between about 12 hours to about 3 weeks. For example, the endotoxins can be processed and expelled within about 12 hours, about 1 day, about 3 days, about 1 week, about 2 weeks, or about 3 weeks.

Endotoxins are notoriously difficult to remove from materials. They are extremely resilient; they are strong, tough and elastic, remain viable after steam sterilization and normal desiccation, and can pass through filters. Research shows that temperatures in excess of 200° C. for up to an hour can be required to remove endotoxin contamination.

As endotoxins are ubiquitous in biological materials, much effort and research has been dedicated to removal and/or inactivation of endotoxins in order to make biological materials useful for medical purposes. Some of the treatment methods that have been researched and employed include heat, acid base hydrolysis, oxidation, ionizing radiation such as gamma-irradiation, and ultra-filtration. These methods have varying ranges of effectiveness, expense, and suitability for particular products.

In some embodiments, the ultra-thin chitosan can be prepared as “excelsior-like” fibrids. Fibrids are short, irregular fibrous shards used in the felt and paper manufacturing industry. Fibrids are manufactured more economically than microfibrils and because of the small dimensions may be more susceptible to both nitrogen plasma depyrogenation and resorption. Chitosan engineering technology and textile expertise is necessary to produce and depyrogenate this ultra-thin chitosan formulation.

The ultra-thin chitosan can be prepared through several methods. In some embodiments, the chitosan can be prepared utilizing methods and techniques as described previously herein with reference to FIG. 1. In some embodiments, chitosan blocks can be formed from physical means or techniques such as compression. In some embodiments, the chitosan blocks can be created by utilizing large chitosan flakes directly obtained from shellfish with multi centimeter dimensions. The chitosan flakes can be plasticized with aqueous organic acids, such as acetic or lactic acid. The chitosan flakes are then compressed and consolidated under vacuum to a density greater than 0.6 g km². This treatment will form chitosan blocks for processing.

In some embodiments, the chitosan blocks can be shredded to shards with specific dimensions of width and thickness. The shredded shards manufacturing process can be analogous to the manufacture of cellulose “wood wool” or excelsior used as “Easter basket grass.” Chitosan shard formulation produced through this method can form chitosan material with an increased surface area than that of chitosan microfibrils. In some embodiments, the increased surface area can produce a chitosan material that can be potentially more vulnerable to nitrogen depyrogenation and resorption. In some embodiments, the chitosan shard formation can be amenable to laparoscopic implantation for internal medical purposes. In some embodiments, the chitosan shard formation formed by the shredded shard manufacturing process can have the necessary flexibility and rigidity for internal medical applications.

The width and the thickness can be varied to produce shards for different sizes and shards ideal for further processing. In some embodiments, the chitosan excelsior or shard can be superfine. The chitosan excelsior or shard can have dimensions of 0.15 mm (0.006 inch) strand thickness and 0.5 mm (0.020 inch) width. In some embodiments, the chitosan shard thickness can be from about 1 μm to 5 μm to about 50 μm, 100 μm, 200 μm, or 250 μm; however, thicker or thinner shards can also be advantageously provided in other embodiments. In some embodiments, the chitosan shard strand width can be greater than or equal to about 0.35 mm or less than or equal to about 0.65 mm. In certain embodiments, the strand width can be from about 0.35 mm, 0.4 mm, or 0.45 mm to about 0.5 mm, 0.55 mm, 0.6 mm, or 0.65 mm; however, wider or narrower shards can also be advantageously provided in other embodiments. The fibrids can have a substantially longer length than width, e.g., a length two times the width, five times the width, ten times the width, or 50 times the width or more.

In certain embodiments, the chitosan materials can be formed through other methods to increase the strength or depyrogenation of the material. The materials can be made from compressed chitosan flakes and/or flake-based chitosan shards. For example, at least two other approaches can be taken to produce an excelsior-like chitosan. In some embodiments, a pulp-like fibrid can be made by the shear induced precipitation of a chitosan solution. The shear induced precipitation can be accomplished by dripping a dilute solution into a stirred coagulant. The solution can be stirred by a shear disc.

In some embodiments, the ultra-thin chitosan can be formed by slitting chitosan film into narrow strips. The term “ultra-thin” as used herein is a broad term, and is to be given its ordinary and customary meaning to a person of ordinary skill in the art (and is not to be limited to a special or customized meaning), and refers without limitation to thicknesses of 0.5 mm or less. The term “narrow strips” as used herein is a broad term, and is to be given its ordinary and customary meaning to a person of ordinary skill in the art (and is not to be limited to a special or customized meaning), and refers without limitation to widths of 1 mm or less. The narrow strips can be of a predetermined dimension. The strips can be of uniform size or the size can be varied. In some embodiments, the narrow strips can be 1 mm wide, 0.5 mm thick and up to 5 cm long. In some embodiments, the chitosan strip width can be greater than or equal to about 0.5 mm or less than or equal to about 1.5 mm. In certain embodiments, the strip width can be about 0.5 mm, 0.75 mm, 1.0 mm, 1.25 mm, and 1.5 mm. In some embodiments, the chitosan strip thickness can be greater than or equal to about 1 μm or less than or equal to about 200 μm. In certain embodiments, the strip thickness can be about 1 μm, 5 μm, 25 μm, 50 μm, 100 μm, 200 μm, or 250 μm. In some embodiments, the chitosan strip length can be greater than or equal to about 1 cm or less than or equal to about 5 cm. In certain embodiments, the strip length can be about 1 cm, 2 cm, 3 cm, 4 cm, and 5 cm.

Several physical forms of a chitosan dressing are available for depyrogenation and endoscopic deployment. In some embodiments, it is necessary to identify an optimal chitosan form compatible with implantation via a laparoscopic port. For example, if the optimal form is a non-woven material that can be furled and unfurled through an endoscope, chitosan textile with shards and/or fibrids can be constructed. For example, in some embodiments, the chitosan textile can be produced by the production of shards from compressed chitosan blocks as described herein. The manufacture of chitosan shards from a compressed chitosan block is a method for producing a chitosan non-woven textile that may be more readily depyrogenated due to the increase in exposed surface area of the shard material. In some embodiments, the production of chitosan shards and/or fibrids can be desirable because of their increased ability to be resorbed after implantation.

It has proven difficult, however, to develop an endotoxin removal or inactivation process (depyrogenation) that is suitable for chitosan as known processes such as contacting the chitosan with a strong base or γ-irradiating aqueous chitosan solutions tends to depolymerize the chitosan, resultingly decreasing the average molecular weight.

In some embodiments, chitosan-based hemostatic textile employ chitosan can have high molecular weight, e.g., greater than 600 kDa. Obtaining such chitosan involves choices and procedures. A source of chitin for use in preparing embodiments of chitosan textiles can be crab shell. Chitin prepared from crab shell, particularly arctic crab shell, generally exhibits a molecular weight that is much higher than the molecular weight of chitin made from shrimp shell. Crab shell chitin also generally exhibits a higher degree of deacetylation than shrimp shell chitin. Crab shell chitin typically exhibits an average molecular weight of from about 600-1,300 kDa. Such high molecular weight chitosan can more readily be processed to form sturdy fibers.

Chitin material for use in preparing chitosan fiber in accordance with some embodiments has a molecular weight of greater than about 600, 650, 700, 750, 800, 850, 900, 950, 1000, 1100, 1200, 1300, 1400, or 1500 kDa or more. In some examples, resulting chitosan fibers have similar molecular weights. The chitosan can have a degree of deacetylation in a range between about 75-90%, between about 80-88%, or between about 80-85%.

In accordance with an embodiment, arctic crab shells such as Alaska snow crab shells are used as the raw material for microfibrillar chitosan. These shells can be washed, crushed, dried, then soaked for 12 hours in 3-5% HCl for 1-2 hours to demineralize and deproteinize the material. The slurry is transferred into a 5% NaOH reactor at 90° C. for another protein removal. Deproteinized crushed shells are washed twice with water until neutral, dried and decolorized again by exposure to ultraviolet light. Another decalcification and deproteinization follows for 12 hours in 3% HCl, followed by 3-5% NaOH 90° C. for another 1-2 hours. The deproteinized, demineralized material is washed by water to neutrality, dried and UV decolorized. At this stage the shell material has been processed to the form of chitin, and has a residual protein level≦0.1%, which is significantly lower than commodity grade chitosan.

To process the chitin to high molecular weight chitosan in accordance with one embodiment, the material is subjected to controlled deacetylation in a 48% NaOH solution at 90° C. for 4 hours. The degree of deacetylation (DA) can be monitored by titration method to 80-88%, or 85% as mentioned above, in order to produce high molecular weight (M.W.) chitosan (M.W.>600 kDa). Also, as noted above, crab shell chitin is unique in providing high molecular weight chitosan. Applicants have determined that high molecular weight chitosan provides a significant advantage for both endotoxin/pyrogen reduction and microfiber production in order to facilitate construction of a chitosan-based hemostatic textile. The processing methods as disclosed herein are also applicable to chitosans of other molecular weights, e.g., less than or equal to 600 kDa.

To process high molecular weight chitosan (in some embodiments a high molecular weight is considered to be ≧600 kDa) or other chitosan in accordance with an embodiment, the chitosan is dissolved in 1% trichloroacetic acid, filtered, deaerated and forced under pressure into a solidifying bath through the pores of a spinning jet (the spinneret pack). Chitosan fibers recovered from the solidified bath are washed, dried, and collected as fibers in a solidifying acetone bath (14° C.). The aperture of the spinning jet can be 0.8 mm (800 microns), hole count 48, and spinning velocity 10 m/min. 20° C. Chitosan fibers from the acetone bath are moved by conveyor belt to a second solidifying bath (methanol at 15° C.). Fibers are maintained in the second solidifying bath for 10 minutes, recovered, and coiled at a velocity of 9 m/min. Coiled fibers are neutralized in a 0.3 gm/L KOH solution for 1 hour before washing with deionized water, then dried, packaged and quarantined until cleared by analysis.

Chitosan processed as just discussed has been analyzed to yield the specifications as depicted in the below table, which specifications conform to the following guidelines: “ASTM F2103-01 Standard Guide for Characterization and Testing of Chitosan Salts as Starting Material Intended for Use in Biomedical and Tissue Engineered Medical Product Applications.”

| Item | Specification |
|---|---|
| Bioburden, aerobic count | A total aerobic count less than 500 cfu/gram. |
| | Total aerobic, fungi, spores and obligate |
| | anaerobes under 1000 cfu/gram |
| Degree of Deacetylation | 85% |
| Average Molecular Weight | 700,000 Daltons |
| pH of H₂O—C₂H₅OH Aq. | 5 ± 0.5 |
| Heavy Metals: Pb, Cr, Hg, | ≦20 ppm |
| Cd, As | <20 ppm total |
| Weight Loss on Drying | <15% |
| Color | White to slight yellow |
| Extractable Material | <0.1% protein |
| Solubility in Acid | <0.5% non-soluble in 1% acetic acid |
| Identity | FTIR |
| Bulk Packaging for | Sealed in metalized foil bags under nitrogen |
| Shipping | |
| Residual Protein | <1% |

Included Specifications after Microfiber,
Non-woven Fabric Production

| | |
|---|---|
| Fiber Denier | Range 9.1-26.9 micron O.D. |
| In vitro adhesion | Adhesive strength (kPa ~ 70-80) |
| Chitosan structure | No change in IR spectrum after UV |

It has been found that high molecular weight chitosan has less of an affinity for endotoxins than low molecular weight chitosan. Thus, although a need to inactivate endotoxins likely still exists, the high molecular weight chitosan is more amenable to successful inactivation treatment.

Chitosan is graded by “purity,” ranging from impure “food” or “commodity grade” to highly purified “medical grade.” To qualify as “medical grade” chitosan endotoxin/pyrogen levels have to be reduced as designated by the FDA and U.S. Pharmacopeia. The endotoxin standards (USP27) for FDA approval of implantable medical devices (chitosan hemostats) are <20 EU (endotoxin units) per device or <0.5 EU/ml in water. Since endotoxin molecular weights vary (10,000 to 10⁶ Da), quantitation is measured as EU, where one EU is equivalent to 100 pg of E. coli lipopolysaccharide (LPS). These levels are typically measured by the Limulus Amoebocyte Lysate (LAL) test.

There is wide variation in endotoxin levels of commercially available, shellfish-sourced “medical grade” chitosan. Table 1 below shows endotoxin levels in commercially available “medical grade” chitosans. The commercially available “medical grade” chitosans as shown in Table 1 have endotoxin levels that do not meet the FDA endotoxin standards for implantable medical devices.

TABLE 1

| | Endotoxin concentration |
|---|---|
| Sample | EU/gm |
| Chitosan glutamate | 247 |
| Glycol chitosan | 311 |
| LMCS (precursor low M.W. chitosan) | 311 |
| ZWC (An/Am = 0.3) | 6,860 |
| ZWC (An/Am = 0.7) | 14,150 |

Endotoxin levels in commercially available “medical grade” chitosans
LMCS = precursor low M.W. chitosan;
ZWC, ZWC Zwitterionic chitosan

In some examples, handling and storage of the manufactured chitosan product is conducted in an endotoxin-reduced, UV irradiated environment. All bags, containers, and storage materials can be pyrogen free and the product is stored and transferred in a nitrogen atmosphere.

In one embodiment, end-product high molecular weight fibrous chitosan fleece was packaged under nitrogen. In some such embodiments, the fleece is packaged in a container made of olefin fibers such as Tyvek™. In some embodiments the packaging comprises a plastic material with or without a thin metalized layer. It is anticipated that other types of packaging may be employed. In some embodiments, however, the packages are sealed, keeping the fleece in an environment of nitrogen gas, and preventing entry by oxygen.

In another embodiment, packages having high molecular weight fibrous chitosan fleece prepared as discussed above and sealed in a nitrogen field such as just discussed can be irradiated with γ-irradiation (CO⁶⁰ source) at 25 kGy over 15 hours. It is anticipated and understood that other doses and intensities of γ-irradiation can be employed. However, Applicants tested chitosan fleece so prepared by implantation into rabbits to monitor the toxic response and thus evaluate the effectiveness of γ-irradiation in inactivating endotoxin contamination in high molecular weight chitosan. Applicants noted the septic response to the γ-irradiated chitosan was markedly less than that of the non-irradiated chitosan as implanted into the same rabbit. More particularly, non-irradiated chitosan exhibited substantial pus formation and localized necrosis and inflammation, while the γ-irradiated sample showed little to none of these effects.

Applicants sent six samples of high molecular weight chitosan samples prepared as discussed above and γ-irradiated under nitrogen for LAL testing, along with six samples that had not been irradiated. The samples were prepared as summarized below:

| Sample | Samples were cut and immersed: | | | |
|---|---|---|---|---|
| Preparation: | Extraction Method: | X | Immersion | Fluid Pathway |

| | |
|---|---|
| No. of Samples: | 6 |
| Total Extraction Volume: | 60.0 mL |
| Static Soak Time: | 60 minutes |
| Extraction Temperature: | 20-25° |

The samples were then tested to detect the concentrations of EUs per device. Since certain properties of endotoxins often interfere with the results of undiluted samples, endotoxins were measured at stepped levels of dilution, with anticipated results becoming more reliable with successive dilutions. The test results follow below:

| | | | |
|---|---|---|---|
| ENDOTOXIN UNITS (EU) | Undiluted | 20.70 | EU/Device |
| PER DEVICE: | 2 fold | 18.40 | EU/Device |
| | 10 fold | 9.77 | EU/Device |
| | 20 fold | 8.60 | EU/Device |

As indicated in the test results, the reliable 10 fold and 20 fold diluted test samples yield levels of EU/Device that are well within the acceptable limits for medical grade, implantable chitosan.

In contrast, the six samples that were NOT irradiated were prepared in a similar manner, yet yielded the following test results:

| | | | |
|---|---|---|---|
| ENDOTOXIN UNITS (EU) | Undiluted | >50.00 | EU/Device |
| PER DEVICE: | 2 fold | 70.00 | EU/Device |
| | 10 fold | 68.80 | EU/Device |
| | 20 fold | 73.00 | EU/Device |

The 10 fold and 20 fold diluted sample tests show levels of EU that are well beyond the acceptable levels of EU for medical grade chitosan. As the only difference in the samples was γ-irradiation in a sealed package in a nitrogen environment, Applicants have concluded that γ-irradiation of chitosan, e.g., high molecular weight chitosan, under these conditions effectively inactivates endotoxins. Additionally, testing of the γ-irradiated chitosan against non-irradiated chitosan for hemostatic efficacy resulted in no detectable difference.

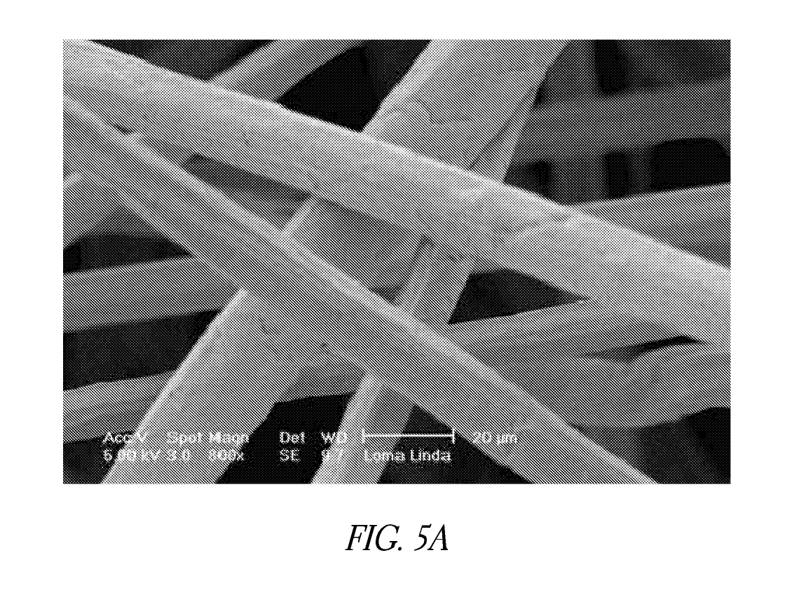

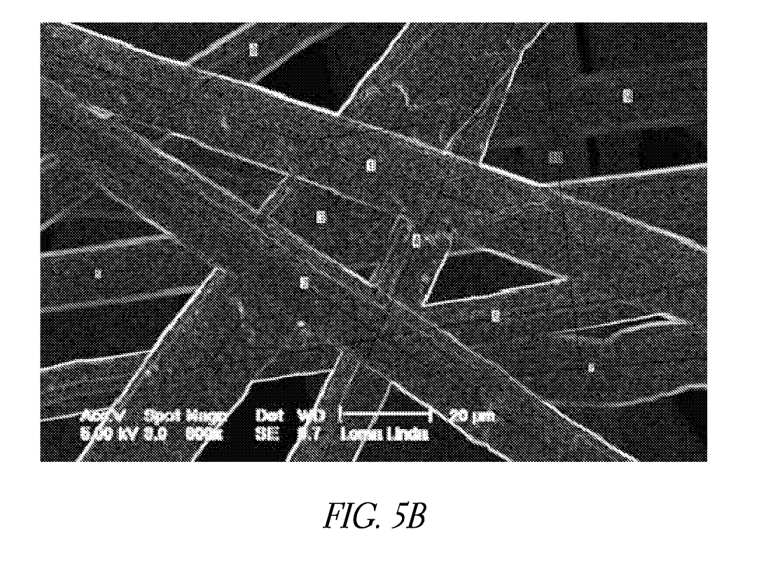

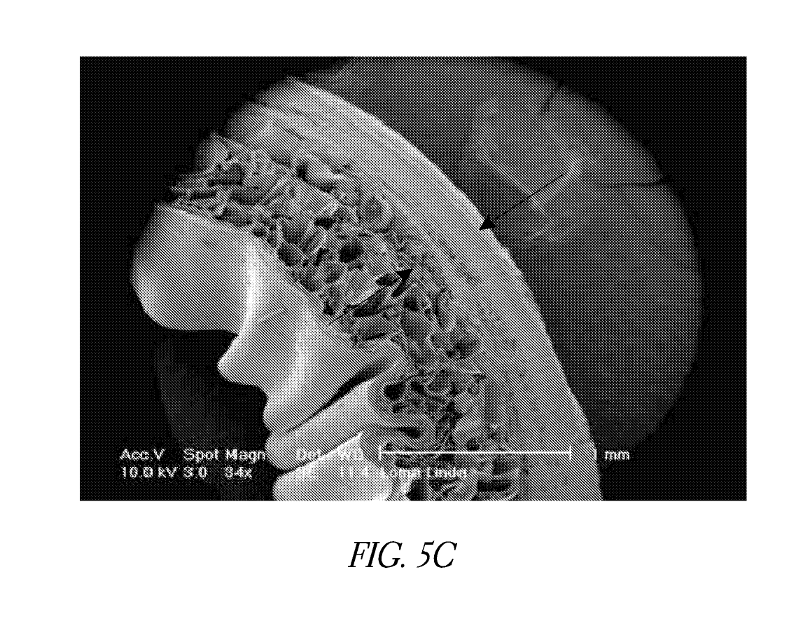

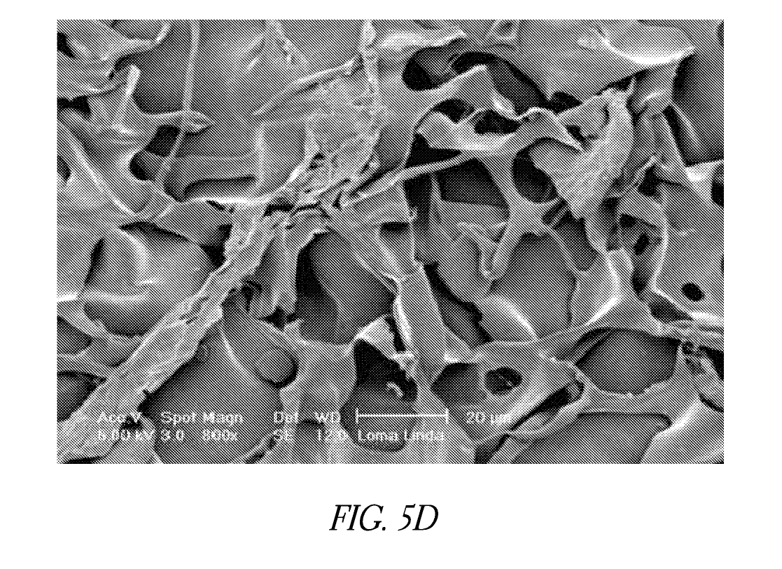

The samples were further investigated to determine whether the γ-irradiation had caused depolymerization and/or otherwise damaged the chitosan fibers. The images in FIGS. 5A and 5B depict Scanning Electron Microscopy (SEM) surface areas of microfibrillar chitosan processed as described above and irradiated as discussed above. FIG. 5A is a SEM of microfibrillar chitosan, mean diameter of fibers 16.7±3.6 μm (range 10-26 μm). FIG. 5B is an edge enhanced image of FIG. 5A, created and analyzed using ImageJ software (ImageJ, NIH). Eleven fibers in the 150×100 μm field of view (FOV) were modeled as cylinders using fiber length and width estimates from the image. The surface area to volume ratio (S/Vₚ) of microfibrillar chitosan using the FOV dimensions and assuming a depth of six times the average fiber diameter (16.7 μm), is 4.7 nm⁻¹. Therefore, a dressing thickness and blood penetration depth of 5 mm, a 1×1×5 mm volume of microfibrillar chitosan presents an estimated surface area of 23.5 μm² to blood products.

### Chitosan Depyrogenation Techniques

The results of the use of γ-irradiation and nitrogen plasma purification techniques in a nitrogen environment on chitosan are discussed in U.S. Pat. No. 8,623,274 and U.S. patent application Ser. No. 14/097,151, filed Dec. 4, 2013 (U.S. Patent Application Publication 2014/0093421). U.S. Pat. No. 8,623,274 and U.S. patent application Ser. No. 14/097,151, filed Dec. 4, 2013 entitled “CHITOSAN-BASED HEMOSTATIC TEXTILE”, are hereby incorporated by reference in their entirety. U.S. Pat. No. 8,623,274 and U.S. patent application Ser. No. 14/097,151, filed Dec. 4, 2013 include information directed to making and using depyrogenated chitosan-based devices by utilizing the techniques of nitrogen plasma and/or gamma irradiation treatment. Additionally, the beneficial properties of depyrogenation treatments and chitosan hemostatic and drug delivery uses are described in more detail in U.S. PCT Application No. PCT/US2014/050188, filed Aug. 7, 2014. U.S. PCT Application No. PCT/US2014/050188, filed Aug. 7, 2014 entitled a “SUBSTANCES AND METHODS FOR THE TREATMENT OF CEREBRAL AMYLOID ANGIOPATHY RELATED CONDITIONS OR DISEASES”, now published as WO 2013/138368, is hereby incorporated by reference in its entirety.

In U.S. Pat. No. 8,623,274 and U.S. patent application Ser. No. 14/097,151, filed Dec. 4, 2013 and experiments described herein, chitosan samples were analyzed to determine whether γ-irradiation had effectively inactivated endotoxins and whether the γ-irradiation had caused depolymerization and/or otherwise damaged the chitosan fibers. It was concluded that γ-irradiation of chitosan under the conditions present in that testing effectively inactivates endotoxins. Additionally, testing of the γ-irradiated chitosan against non-irradiated chitosan for hemostatic efficacy resulted in no detectable difference. Further, the irradiated chitosan fibers were structurally intact, and maintained a high surface area that was available for interaction with blood. It was concluded that the irradiation under the listed conditions caused no significant depolymerization and/or reduction in molecular weight of the chitosan fibers. Accordingly, it can be advantageous to subject chitosan fibers to γ-irradiation in either a presence or an absence of nitrogen plasma.

The chitosan fibers prepared as discussed above have a relatively high nitrogen content. Applicants have determined that treating such fibers in conditions conducive to ionization of nitrogen is especially beneficial in inactivating endotoxin without substantially damaging the chitosan fiber structure or the efficacy of the fibers in prompting hemostasis. More particularly, in some embodiments, chitosan is subjected to a treatment that increases the quantity of amino groups in and around fibrous chitosan. In other examples, the chitosan can be subjected to a treatment that creates nitrogen-based free radicals, so as to inactivate endotoxin and simultaneously increase one or more of wetability, hydrophilicity, and mucoadhesion.

The chitosan materials have a relatively high nitrogen content. In U.S. Pat. No. 8,623,274 and U.S. patent application Ser. No. 14/097,151, filed Dec. 4, 2013 and experiments described herein, chitosan samples were analyzed to determine whether nitrogen plasma had effectively inactivated endotoxins and whether the nitrogen plasma had caused depolymerization and/or otherwise damaged the chitosan fibers. It has been determined that treating such materials in conditions conducive to ionization of nitrogen is especially beneficial in inactivating endotoxin without substantially damaging the chitosan material structure or the efficacy of the chitosan in prompting hemostasis and bioadhesion. More particularly, in some embodiments, chitosan can be subjected to a treatment that increases the quantity of amino groups in and around the chitosan material, so as to inactivate endotoxin and simultaneously increase one or more of wettability, hydrophilicity, and mucoadhesion. In some embodiments, chitosan can be subjected to a treatment that creates nitrogen-based free radicals which can also inactivates endotoxins and simultaneously increases one or more of wettability, hydrophilicity, and mucoadhesion.

In some embodiments, a thin paper-like sheet can be utilized as an end product for internal and/or external purposes to promote wound healing and/or to act as a vehicle for drug delivery. In other embodiments, the thin paper-like sheets can be combined with other sheets of the same chitosan fibrid material and/or different material to form a thicker end product including a network of chitosan fibrids. However, in other embodiments, a thicker end product can be made by making a network of chitosan fibrids, chitosan shards, or a combination of both. In some embodiments, the network of ultra-thin chitosan can include a fleece-like, fabric-like, and/or assembly of chitosan shards, films, ribbons, fibrids, and/or other forms of chitosan material as described herein and in International Patent Application No. PCT/US2015/025605, filed Apr. 13, 2015, and directed to a “COMPOSITION, PREPARATION, AND USE OF CHITOSAN SHARDS FOR BIOMEDICAL APPLICATIONS” and International Patent Application No. PCT/US2015/035885, filed Jun. 15, 2015, and directed to a “COMPOSITION, PREPARATION, AND USE OF CHITOSAN HEMOSTAT FOR LAPAROSCOPIC PARTIAL NEPHRECTOMY.” The entirety of these applications is hereby incorporated by reference. Additionally, the chitosan end product can contain medicinal agents and/or other active agents to assist in wound healing or drug delivery as described in detail herein. In some embodiments, similar to chitosan of fibrous materials, the chitosan fibrids can also be constructed as a textile in a puff, fleece, fabric or sheet form.

For example, a chitosan material is treated with an ionized nitrogen gas, more specifically a nitrogen-based plasma, under ambient temperature, so as to effectively inactivate endotoxins on chitosan without negatively affecting the efficacy or molecular weight of the chitosan.

Gas plasmas, for example, can be used for sterilization in the food and beverage bottling industries inactivating bacteria, bacterial spores, viruses, fungi, prions and pyrogens. The treatment of chitosan by non-thermal gas plasma technique provides significant advantages over conventional sterilization methods. For example, non-thermal atmospheric nitrogen plasma (NtANP) gas plasmas do not degrade thermo-labile chitosan in contrast to electron beam sterilization methods.

In some embodiments, non-thermal atmospheric nitrogen plasma may be the ideal reagent for depyrogenating chitosan since the process not only sterilizes, but increases surface mucoadhesivity by addition of elemental nitrogen to chitosan surfaces. Surface modification of chitosan membranes with an increased incorporation of nitrogen and oxygen groups has been established with N₂ plasma treatment. In some embodiments, the surface modification can enhance surface hydrophilicity and bioadhesion. Accordingly, it can be advantageous to subject chitosan fibers to nitrogen plasma in either a presence or an absence of γ-irradiation.

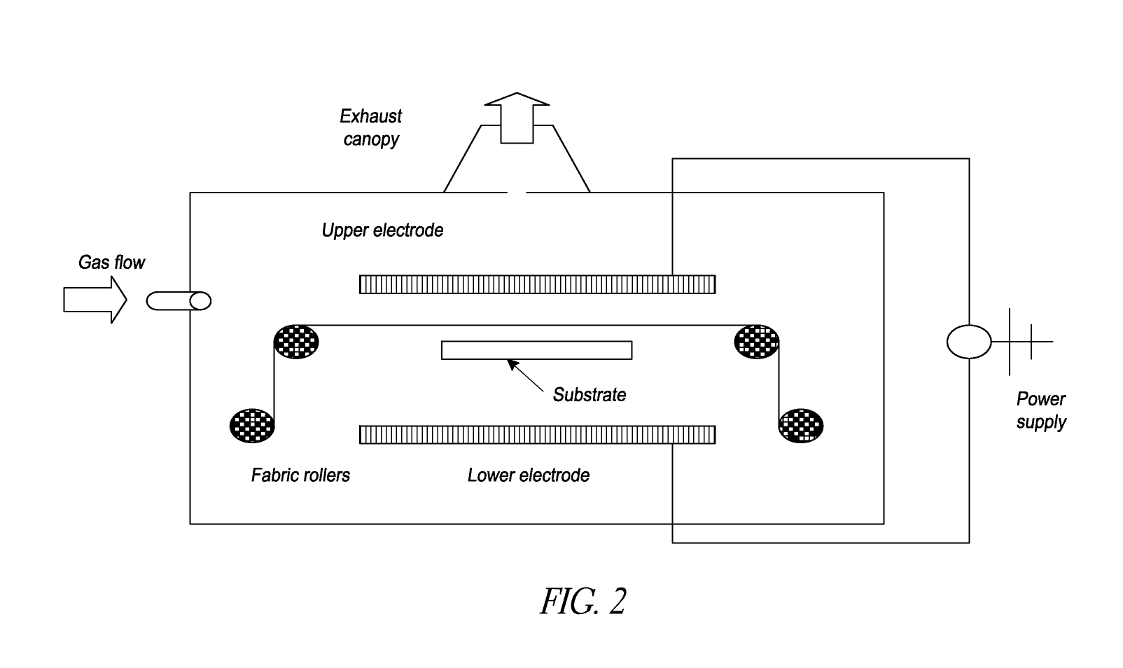

FIG. 2 illustrates a schematic of the North Carolina Atmospheric Plasma System (NCAPS) at North Carolina State University which can be utilized to treat chitosan materials. In some embodiments, the nitrogen plasma instrument can be used to treat the chitosan material as described herein.

The schematic of the low temperature nitrogen plasma sterilization illustrated in FIG. 2 shows the simple, high throughput system. Temperature measurements are controlled by a TEFLON® coated thermo-coupler moderated by an analog-to-digital converter. In some embodiments, experimental conditions including time and frequency of plasma exposure and voltage between electrodes can be controlled and varied with this instrument.

In some embodiments, an optimal nitrogen plasma dosimetry can achieve a chitosan with EU levels<20 EU/gm. For example, a “sub-sterilization incremental dose” protocol with variations in power, frequency and plasma exposure time can be applied to our selected chitosan hemostat to allow survival curves to be calculated for residual endotoxin (EU/g) and bacterial bioburdens (Colony Forming Units (CFUs/gm). 50 CFUs and EUs plotted on a log vertical scale and plasma exposure (time power) on the linear horizontal scale enable calculation of the log [N(t)/No]=k·t relationship (No is initial concentration of CFUs/EUs, Nt the concentrations found at given time and power, k is the endotoxin “death rate” constant, t=Time). To reduce the original CFU/EU levels by 90% is one log 10. A 6 log 10 reduction in EU is necessary to secure adequate endotoxin reduction for implantation.

An example of chitosan endotoxin reduction with nitrogen plasma that has been observed with these techniques is given in Table 2. Conditions used in the laboratory for this nitrogen plasma treatment are as follows: frequency; 1.373 kHz with voltage across the plates at 6.3 kVrms and 7.6 KVmax.

TABLE 2

| Material | EU/gm |
|---|---|
| Chitosan/Foreign Source (FS) before plasma | 620.5 |
| *Chitosan FS v-irrad. + N2 plasma (5 min) | 52.8 |
| **Chitosan FS Fabric y-irrad. + N2 plasma (10 min) | 12.7 |
| ***Shrimp Lotox Chitosan (powder) (HemCon) | <65 |

*Endotoxin levels were determined by Steri-Pro Labs, Ontario, CA. Endotoxin levels are now determined in Dr. Kirsch's laboratory and crosschecked in the Steri-Pro Labs - see B.4.
**FDA endotoxin requirements for an implantable medical device [symbol] 20 EU/gm or device).
***Shrimp Lotox ™, “ultrapure chitosan,” from Syndegen, Claremont CA (material not available for commercial sale), molecular weight (M.W.) 160-312 kDa.

Table 2 displays chitosan endotoxin levels before and after nitrogen plasma treatment. Chitosan non-woven textile from a foreign source (FS) and previously gamma irradiated were assayed in this experiment.

In some embodiments, when chitosan is treated with nitrogen plasma the effects of nitrogen plasma on chitosan materials have established that despite shallow surface penetration of N2 plasma reactants (NO, OH radicals, N metastables) there are no alterations of hemostatic functional capability.

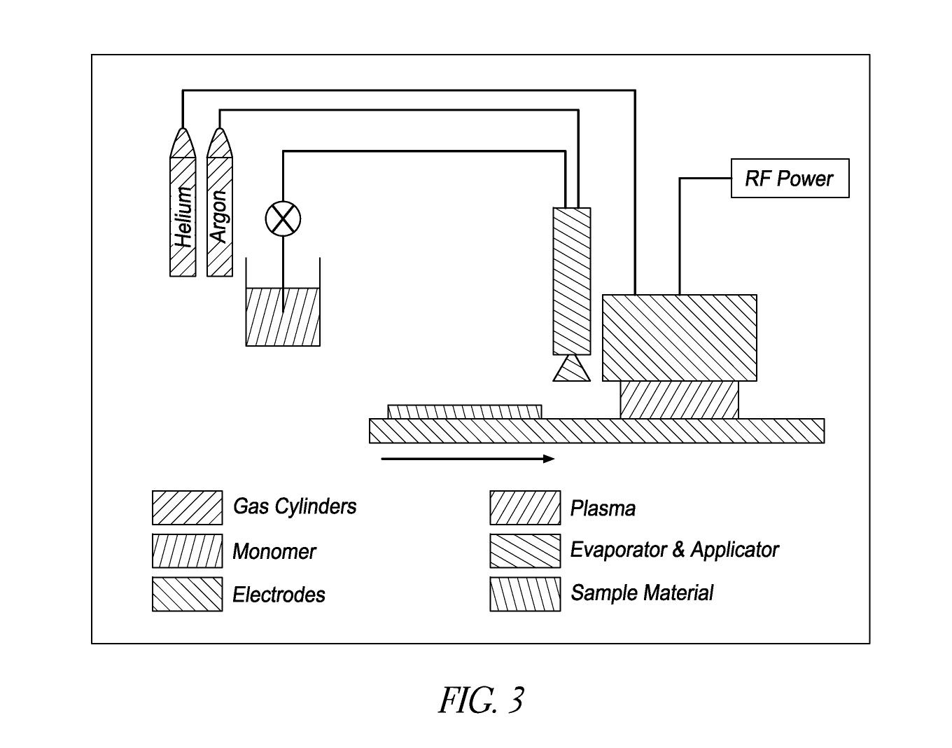

In some embodiments, plasma treatment can also be carried out using, for example, an e-Rio™ atmospheric pressure plasma system APPR-300-13 available from APJeT Inc. The machine uses RF electric fields, 1300 W @ 27 MHz RF/1 mm gap, to produce a unique, non-thermal, glow-discharge plasma that operates at atmospheric pressure with a cooling requirement of 1 gpm @ 20 psi max. In some embodiments, non-atmospheric N₂ plasma instruments can be used. FIG. 3 is a schematic depiction of one embodiment of a plasma treatment assembly. In some embodiments, the plasma assembly can include an evaporator and applicator. The evaporator can be a heated assembly that vaporizes a monomer that is to be applied to the chitosan samples. Heat is regulated by a logic controller that is connected to a thermo-coupler attached to the evaporator. The applicator acts as a heated nozzle to apply vaporized monomer to the fibrous chitosan sample. The heat maintains the vapor property of the monomer. Heat can be regulated by a logic controller that is connected to a thermo-coupler attached to the applicator.

It is to be understood that multiple methods and assemblies for plasma treatment of chitosan can be employed. Plasma treatment and the accompanying depyrogenation techniques as described herein can be applied to chitosan material or the derivatives thereof. The plasma source can be various gases used in combination or individually. The plasma source creates a highly reactive chemical environment and/or a highly energized environment to purify and depyrogenated the chitosan material. The highly reactive chemical environment can allow the plasma treatment to physically and/or chemically alter the chitosan material. The plasma treatment affects the physical properties of the chitosan material. The ions within the plasma chamber accelerate to disturb and remove contaminates from the surface of the material. The chemical effects from the plasma treatment can affect more than the surface of the material and provide additional purification of the material. The chemical effects typically occur at higher temperatures, higher energy levels, higher pressures, or after a longer period of treatment. Therefore, the chemical plasma treatments require more extreme conditions and therefore, have a higher likelihood of denaturing or altering the properties or characteristics of the treatment material which can reduce or alter the beneficial properties of the chitosan material for hemostatic and/or drug delivery purposes. Additionally, the packaging and preparation of the material for distribution and sale can also be done under sterilization conditions as described herein. For example, chitosan materials can be treated under a nitrogen plasma and then packaged under nitrogen gas. In some embodiments, relatively large quantities of chitosan materials are treated under nitrogen plasma and are then divided into individual doses and packaged separately. In still other embodiments, chitosan can be partially packaged, such as enclosed within a package having an unsealed opening, plasma-treated in the partially packaged condition, and the package may be fully sealed in the plasma treatment zone or a nearby nitrogen field. In some embodiments, chitosan materials can be packaged in Tyvek® pouches under nitrogen gas, sealed, and subsequently treated with plasma.

In further embodiments, the chitosan material can be packaged prior to plasma treatments. In some embodiments, the chitosan material is sealed in a nitrogen field, and can be prepared substantially as discussed above. In some embodiments, the RF power activates the nitrogen within the packaging, which is believed to create nitrogen-based free radicals that contribute to deactivation of the endotoxin. Of course, it is to be understood that various types and configurations of assemblies and apparatus may be used for the plasma treatment.

Embodiments discussed above have described treating chitosan materials in a nitrogen field involving plasma, γ-irradiation, or the like. In other embodiments, other methods and apparatus that will increase the concentration of amino groups on and around the chitosan can be employed. Such methods can additionally provide nitrogen-based free radicals. Such methods may involve other types of irradiation, as well as variations in power, duration, and the like as compared to the examples specifically discussed herein.

The type of gas chosen for plasma treatment can have varying effects on the physical and chemical plasma treatment of the material. The gases can include oxygen, nitrogen, ammonia, hydrogen peroxide, nitrous oxide, air, and/or other gases known in the art for plasma treatment and sterilization. The gases can be selected to provide the appropriate or beneficial modification of the material. For example, oxygen can be used to denature proteins and the by-products produced are carbon dioxide and water. The reactive oxygen species react with organic contaminants to form the H₂O, CO, CO₂, and lower molecular weight hydrocarbons. The resulting compounds have relatively high vapor pressures and are evacuated from the chamber during processing. The by-products are non-reactive and prevent additional unwanted alterations during or after treatment. In another example, nitrogen gas can be utilized to purify the chitosan material as well as allow the atomic nitrogen to alter the surface of the chitosan material to increase mucoadhesivity of the material as well as create other beneficial properties of the chitosan material for hemostasis or drug delivery as described herein. Further, the nitrogen gas is less oxidative then other gases including oxygen.

Additionally, the pressure and temperature within the plasma chamber can be varied to provide physical and chemical treatments of the materials for the selected or optimal material characteristics desired. The pressure and temperature can be chosen to provide the optimal depyrogenation while maintaining the beneficial characteristics of the chitosan materials. For example, the pressure and temperature within the chamber can provide purification of the chitosan material while maintaining the flexibility of the material as well as the bioabsorbability, mucoadhesion, and hemostatic properties. The pressure can be low or high pressure.

The pressure can be altered during treatment of the material. The pressure can be altered between high or low pressure one or more times throughout the plasma treatment. For example, the plasma can be initially treated at low pressure for a period of time followed by a second period of treatment at high pressure and vice versa. Further, in another embodiment, the plasma can be pulsed on and off throughout the treatment time. The chitosan material can remain within the plasma chamber throughout the entirety of the treatment time. In other embodiments, the chitosan material can be removed from the treatment chamber when the plasma treatment is turned off.

The plasma can be treated at low pressure. The low pressure allows plasma uniformity within the chamber and can allow for better control of the environment within the chamber. Additionally, the low pressure and control provide a reproducible treatment environment where all parameters can be controlled and/or maintained.

In some embodiments, high pressure can be used within the chamber. The high pressure can create a harsher environment than low pressure environments. The high pressure can be utilized to assist in purification by providing a more reactive environment. Therefore, high pressure may be beneficial but could be coupled with less treatment time and/or a lower temperature to prevent the material characteristics from being affected or denaturing of the material.

The plasma chamber can be operated at high frequencies typically in the measures of Hz to kHz to greater than MHz range. In some embodiments, the chamber can be operated at low pressure with a frequency at about 13.56 MHz, applied at a few hundred watts. In some embodiments, the chamber can be operated at around 30 Hz. In some embodiments, less than or equal to about 300 Watts to greater than or equal to about 600 Watts can be used. In some embodiments, about 300 Watts, about 400 Watts, about 500 Watts, or about 600 Watts can be used.

Additionally, the duration and time of plasma exposure can be varied for optimal time or periods of time for purification and/or dyprogenation. The time period of treatment can be between less than or equal to about 15 minutes to greater than or equal to about 120 minutes. In some embodiments, the time period of plasma treatment within the chamber can be about 15 minutes, about 30 minutes, about 45 minutes, about 60 minutes, about 75 minutes, about 90 minutes, about 105 minutes, and about 120 minutes. In some embodiments, the material can be treated in a straight treatment time with continuous plasma exposure throughout the entire period of time. In other embodiments, the material can be maintained in the chamber throughout the entire period of time but the plasma is applied to the chamber in a pulsation pattern as described herein or known in the art. Further, in additional embodiments, the material can be removed from the plasma chamber at set intervals during the period of treatment. Additionally, in other embodiments, the material can be treated with a combination of pulsation of plasma, removing the material from the chamber at set intervals, and/or treating the material in straight intervals. For example, the material can be treated for 90 minutes total. Within those 90 minutes, for the first 16 minutes the material is treated in 2 minute intervals with the material removed from the chamber every 2 minutes. The interval treatment can be followed by 30 minutes of straight plasma exposure. Additionally, in some embodiments, if necessary, for the last 44 minutes the plasma can remain in the plasma chamber but be treated with a pulsation of plasma throughout the time period. As another example, the material is treated for 46 minutes. Within those 46 minutes, for the first 16 minutes the material is treated in 2 minute intervals with the material removed from the chamber every 2 minutes. The interval treatment can be followed by 30 minutes of straight plasma exposure.

The temperature used can be varied to achieve optimal purification or dyprogenation. The temperature can be maintained throughout the treatment time. In one embodiment, the temperature can be maintained at about 70° C. The temperature can be less than or equal to about 50° to greater than or equal to about 100° C. The temperature can be about 50°, about 60°, about 70°, about 80°, about 90°, and/or about 100° C. In other embodiments, the temperature can be varied during the treatment period.

The position of the chitosan within the plasma chamber can alter the intensity of the plasma on the chitosan material. The chemistry of the plasma changes with distance from the discharge of the plasma from the nozzle within the chamber. Additionally, the positioning within the chamber can be controlled by the type of treatment chamber used. The chamber can contain shelves within the chamber. The shelves can be arranged in various configurations and the chamber can contain one or more shelves. The material can be turned over or rotated in the chamber during breaks between the plasma treatment. The rotation or turning can allow for more even treatment of the material, for example, the top and bottom of the material.

In other embodiments, the chamber does not contain shelves but instead the entire chamber can be rotated or contain an interior compartment within the chamber that rotates to allow the material within the chamber to tumble in the chamber throughout the plasma treatment or during a portion of it. The tumbling of the material within the chamber can allow for a more even plasma treatment of the material. Moving the sample during plasma treatment ensures all surfaces of the material receive equal plasma treatment. The chitosan material can be contained in an interior compartment including, for example, a bag or foil container. The plasma treatment could be carried out using a plasma machine similar to those available at PVA-Tepla America, Inc. in Corona, Calif.

In some embodiments, the material can be treated with gamma irradiation in addition to the plasma treatment as described herein. The gamma irradiation treatment can take place before or after the plasma treatment.

In accordance with yet further embodiments, chitosan materials are treated using both plasma and a nitrogen field and γ-irradiation. In some embodiments the chitosan is first treated with γ-irradiation and then treated under the plasma. In other embodiments the order is reversed.

For example, samples of fibrous high molecular weight chitosan were treated. The chitosan had a molecular weight about 700 kDa and a degree of acetylation of about 85%. These samples had been sealed in packages and in a nitrogen field, by first γ-irradiating the packaged samples at a level of 25 Gy, and then plasma treating the still-packaged samples. The treated samples were then subjected to LAL testing. A sample so treated under plasma for about 5 minutes was tested to have 9.6 EU/device, and 52.8 EU/gm based on a 20-fold dilution. A sample so treated under plasma for about 10 minutes was tested to have 2.3 EU/device, and 12.7 EU/gm based on a 20-fold dilution.

In some embodiments described above, chitosan is treated with an acetic acid solution so as to promote adhesion. In further embodiments, chitosan is not treated with acetic acid, and instead is subjected to γ-irradiation in a nitrogen field, nitrogen-gas based plasma treatment, and/or another treatment method that increases the concentration of amino groups on and around the chitosan so as to increase wettability, hydrophilicity and mucoadhesion without exposure to the acetic acid after being formed into a fleece.

It is to be understood that further treatments may enhance hemostatic properties of chitosan-based materials. For example, in one embodiment, chitosan materials are soaked in alcohol, for about an hour. In experiments, such a treatment caused the chitosan materials to be much whiter, but with no structure change of the chitosan fiber. The total bacterial count of the chitosan material was also reduced. Such treated materials can then be further treated using γ-irradiation, plasma, or both.

In some embodiments, the endotoxin levels in the chitosan before electron beam sterilization (25 K Grey) are 29 EU/g. After electron beam sterilization the chitosan can have EU levels about 2 EU/g. However, the chitosan is oxidized which causes a reduction of mucoadhesion, molecular weight (MW), and solubility. Nitrogen plasma exposure results in surface nitrogenation of chitosan that may increase bioadhesive, hemostatic and anti-microbial activity.

In contrast to electron beam, non-thermal atmospheric nitrogen gas plasmas do not degrade thermo-labile chitosan and are the most efficient, least material-damaging reagents. Nitrogen plasma may be the ideal reagent for depyrogenating chitosan since it does not affect physical and functional properties and may, in fact, increase mucoadhesivity by addition of elemental nitrogen to chitosan surfaces.

Various chitosan materials can be subjected to the nitrogen plasma and/or gamma irradiation treatments described herein. Chitosan can be utilized as a hemostat in various compositions and forms. In some embodiments, the form of the chitosan material can have particular advantages that are suitable for different medical purposes, including the use of the chitosan material internal and topical. Examples of various chitosan materials or forms are described herein. For example, FDA-approved topical chitosan hemostats can include lyophilized flakes, granular powder, microfibrillar, high molecular weight chitosan, and microfibrillar, non-woven textiles. In certain embodiments, the chitosan materials for internal and topical use can include chitosan powder, shards, films, ribbons, fibrids, sponges, flakes, slurries, microparticles, nanoparticulates, gels, solutions, aerosols, and/or other forms of chitosan material as described herein.

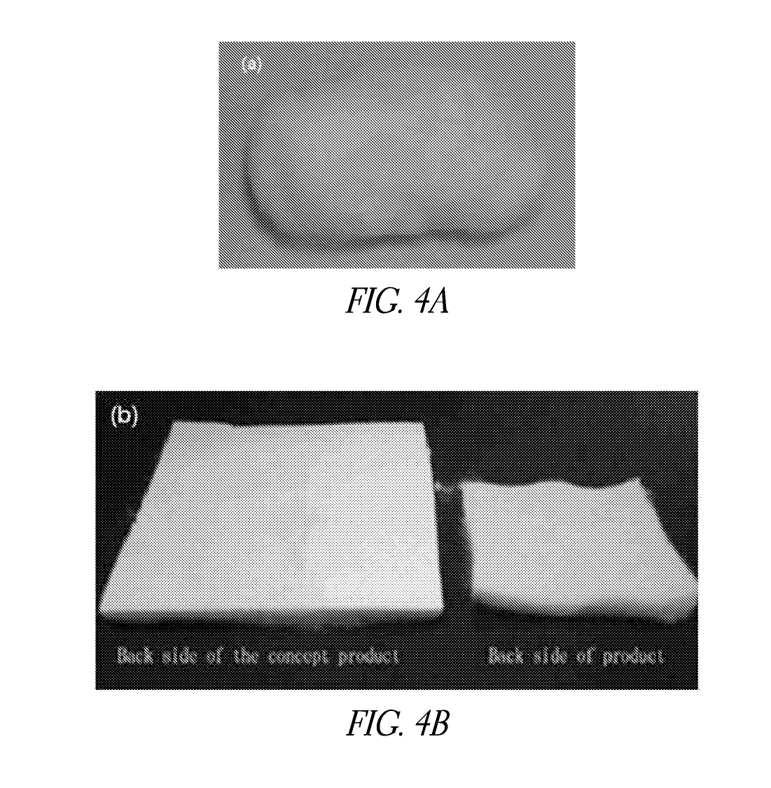

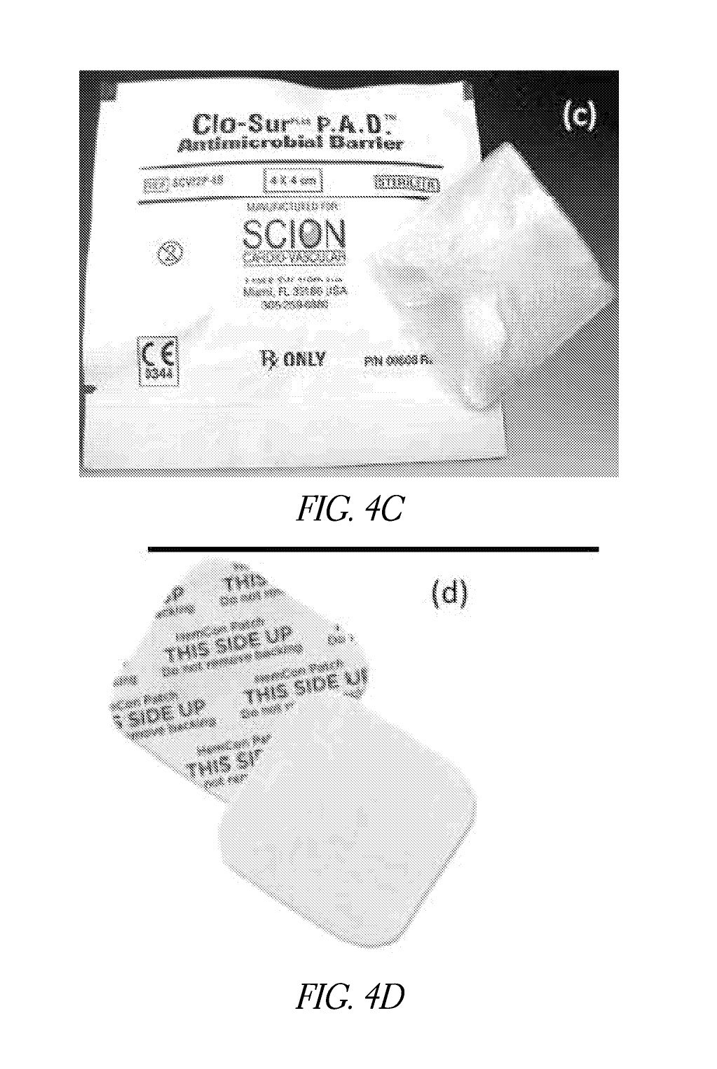

FIGS. 4A-4D illustrate commercially available “medical-grade” forms of chitosan. The medical grade forms of chitosan can vary from fleece alone as in FIG. 4A or fleece compressed into a non-woven fabric or felt as illustrated in FIG. 4B. In some embodiments, the chitosan can be in the form of a lyophilized pads as shown in FIG. 4C. FIG. 4D illustrates a medical grade chitosan in the form of a lyophilized dressing.

In some embodiments, a chitosan fleece can be deployed through endoscopic deployment. The flexibility of materials such as chitosan fleece, non-woven textiles and pads provide ease of deployment through an endoscopic port. In some embodiments, a non-woven fabric or pad can be readily deployed via an endoscopic port whereas a lyophilized product lacks flexibility. In some embodiments, chitosan shards, films, ribbons, fibrids, gels, powders, nanoparticles, microparticles, and/or other forms of chitosan material as described herein can be more readily deployed through an endoscope. Therefore, the choice of chitosan end product will vary depending on the intended use.

In some embodiments, these various chitosan materials can be subjected to the same purification treatments. In some embodiments, the depyrogenation of the chitosan material with nitrogen plasma and/or gamma irradiation techniques as described herein have proven to achieve acceptable levels of endotoxin purification to be utilized in internal application. For example, the depyrogenated chitosan fleece and/or non-woven fabric or pad material can be delivered through an endoscopic port to a surgical site and used or implanted internally to control bleeding, as a therapeutic agent, and/or as a drug delivery vehicle. In another example, depyrogenated chitosan powders and/or flakes can be disposed in a viscous solution or gel and delivered through an endoscopic port to a surgical site and used or implanted internally to control bleeding, as a therapeutic agent, and/or as a drug delivery vehicle.

The hemostatic action of chitosan is based on both tissue bioadhesion and condensation of red blood cells and other formed elements to create a bleeding barrier. Chitosan's glucosamine residues confer a robust surface of positive amine groups binding to tissues' surface negative sialic acid charges. Hypothesized mechanisms for chitosan's bioadhesion are: electronic, adsorptive, wetting, and diffusion. Bioadhesion, whether due to electrostatic, hydrogen bonding, or van der Waals intermolecular interactions creates a hemostatic barrier without activation of the intrinsic or extrinsic coagulation cascade. Chitosan promotes wound healing by activation of macrophages, cytokines, and inhibition of gram-negative bacteria.

### Surface Treatment

The treatment of chitosan materials with nitrogen plasma and gamma irradiation techniques can provide purification of the chitosan material but also the hemostatic advantages as discussed above. Nitrogen plasma acts on the surface of the chitosan material. It has been found that the greater the surface area of the chitosan expose during treatment the more effective and efficient the depyrogenation techniques. In some embodiments, it is advantageous to provide chitosan material with a greater exposed surface area and thereby providing a more readily depyrogenated chitosan material.

The fine structural differences between microfibrillar chitosan fleece and lyophilized chitosan flakes are shown in scanning electron micrographs of FIGS. 5A-D. The microfibrillar chitosan and lyophilized chitosan flakes were studied to determine the effect of plasma mediated surface depyrogenation in comparison to the enhanced surface area of the microfibrillar chitosan.

FIGS. 5A-D provide Scanning Electron Microscopy (SEM) images for comparison of the surface areas for microfibrillar chitosan and a HemCon® lyophilized dressing. FIG. 5A shows a SEM of microfibrillar chitosan with a mean diameter of fibers at 16.7±3.6 μm (range 10-26 μm). FIG. 5B shows an edge enhanced image of FIG. 5A, analyzed using ImageJ software. Eleven fibers in the 150×100 μm field of view (FOV) were modeled as cylinders using fiber length and width. The surface area to volume ratio (S/Vₚ) of microfibrillar chitosan is 4.7 nm⁻¹, thus having a blood penetration depth of 5 mm. A 1×1×5 mm volume of microfibrillar chitosan presents a surface area of 23.5 μm² to blood products. The SEM of a HemCon® lyophilized dressing is in FIG. 5C and FIG. 5D. As shown in FIG. 5C, the HemCon® lyophilized dressing exposes a smaller chitosan surface compared to microfibrillar chitosan. Arrows denote the non-stick side of the dressing. To account for the convoluted surface of HemCon® lyophilized dressing, a surface area gain of 50% is assumed and the S/Vₚ is 4.1 nm⁻¹. A 1×1×0.365 mm volume of HemCon® lyophilized dressing presents a surface area of 0.0015 μm² to blood. The ratio of microfibrillar chitosan surface area to the HemCon® lyophilized dressing in a 1×1 mm patch is 23.5/0.001 or 15,667. The enhanced surface area of microfibrillar chitosan may account for enhanced adhesion, hemostasis, and more effective plasma mediated surface depyrogenation. In some embodiments, it can be effective to increase the surface area of the chitosan material to further enhance the characteristics of the chitosan material, including the effectiveness of depyrogenation techniques.

Chitosan can be pre-treated or specially prepared to increase the effectiveness of nitrogen plasma and gamma irradiation treatment. Because nitrogen plasma can act on the surface of any physical form of chitosan, an increase in surface area to volume ratio can allow for more effective depyrogenation of the chitosan material. In some embodiments, the flexibility required for laparoscopic implantation and internal surgical placement may alter the form of chitosan material used. For example, ultra-thin chitosan materials or chitosan powders or granules formed into solution can be appropriate for laparoscopic implantation due to the durability and flexibility of the material. The extent of depyrogenation of certain forms of chitosan and the required flexibility of the material are characteristics that can direct the shape and/or structure of the chitosan material utilized.

In some embodiments, achieving the targeted endotoxin reduction may require producing and treating ultra-thin chitosan or chitosan powder or granules. The chitosan material can be easier to purify or depyrogenate with surface active agents such as nitrogen plasma. In some embodiments, with ultra-thin chitosan, full sample penetration or substantially full sample penetration by the nitrogen plasma reactants can be achieved. Additionally, in some embodiments, the ultra-thin chitosan can be a strong and durable material that is flexible and malleable but retains continuity so that it can be moved as a unit and doesn't break apart when manipulated during use.

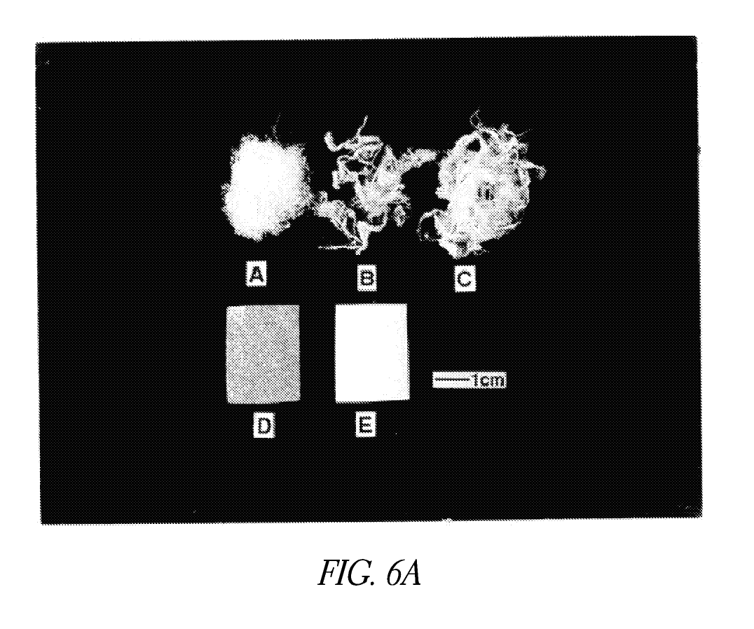

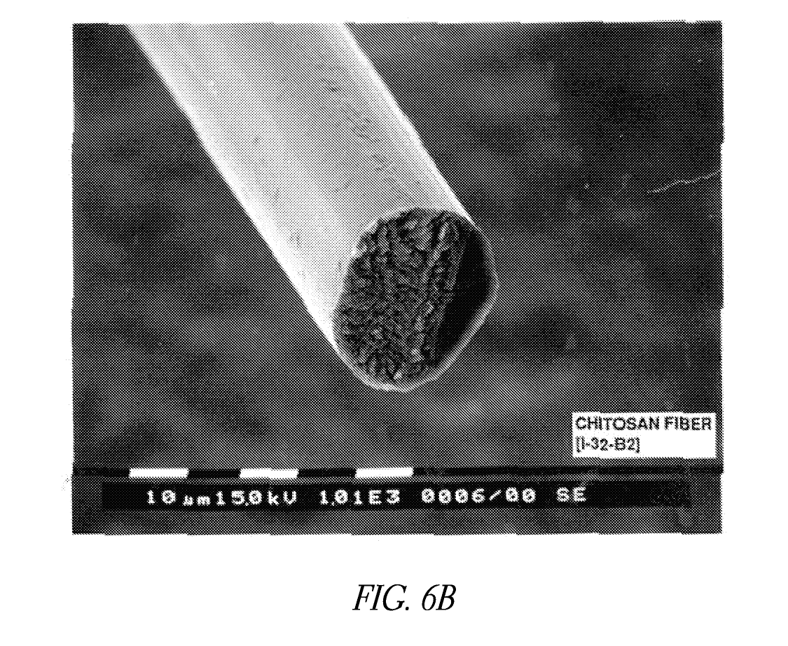

FIG. 6A illustrates embodiments of solution processed chitosan fibrids. The chitosan fibrids can have different appearances and different configurations depending on the solution used to process the fibrids. Chitosan fibrids can be processed in varying processing methods including direct shear precipitation, water processing, alcohol processing, and/or other processing methods known in the art. The different chitosan fibrid end-products of the varying processing solutions are shown in FIG. 6A(a)-6A(e). The embodiments shown in FIG. 6A(a) are chitosan fibrids made by direct shear precipitation. Water processed fibrids have the appearance of the thin narrow strips as shown in FIG. 6A(b). The water processed fibrids of FIG. 6A(b) can be formed into a thin paper-like sheet structure as shown in FIG. 6A(d). FIG. 6A(c) shows alcohol processed and FIG. 6A(e) shows the paper-like sheet obtained from the fibrids of FIG. 6A(c). FIG. 6B illustrates a conventional wet spun chitosan filament. The white scale bar depicted in FIG. 6B represents 10 microns.

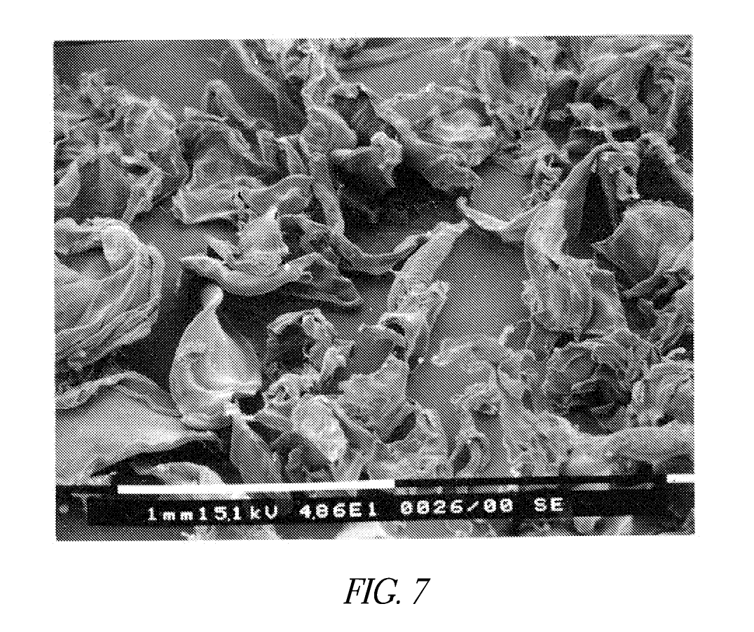

FIG. 7 illustrates chitosan fibrids that are directly obtained by shear induced precipitation of chitosan from solution as shown in FIG. 6A(a). The scale bar in FIG. 7 represents 1 mm. The chitosan fibrids as shown in FIG. 7 illustrate the size and exposed surface area of the chitosan fibrid material. The chitosan fibrid material is thinner than the chitosan fibers produced by conventional wet spinning methods and contain a larger exposed surface area. Due to these features, the ultra-thin chitosan material can be more readily depyrogenated with surface acting agents such as nitrogen plasma treatment and gamma irradiation. In some embodiments, the ultra-thin chitosan can be depyrogenated to allow for endotoxin reduction to levels acceptable for internal medical applications while maintaining the hemostatic and mucoadhesion properties of other chitosan materials.

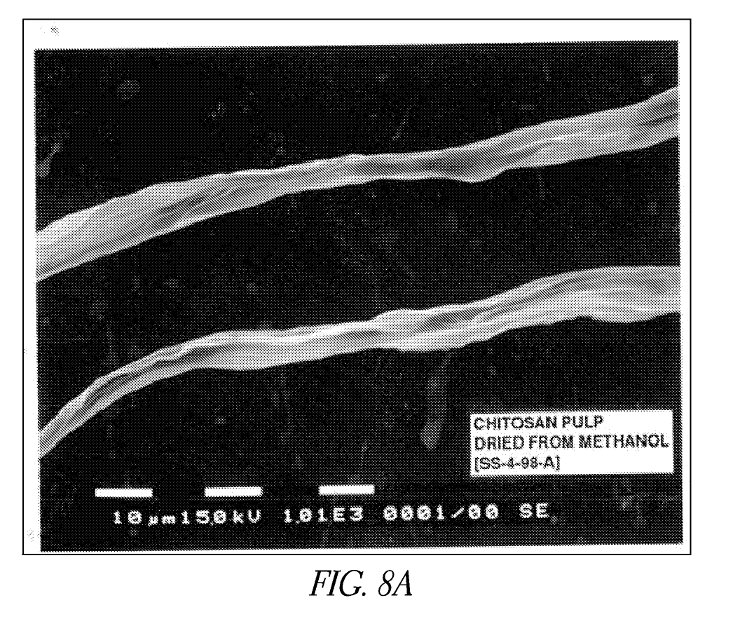

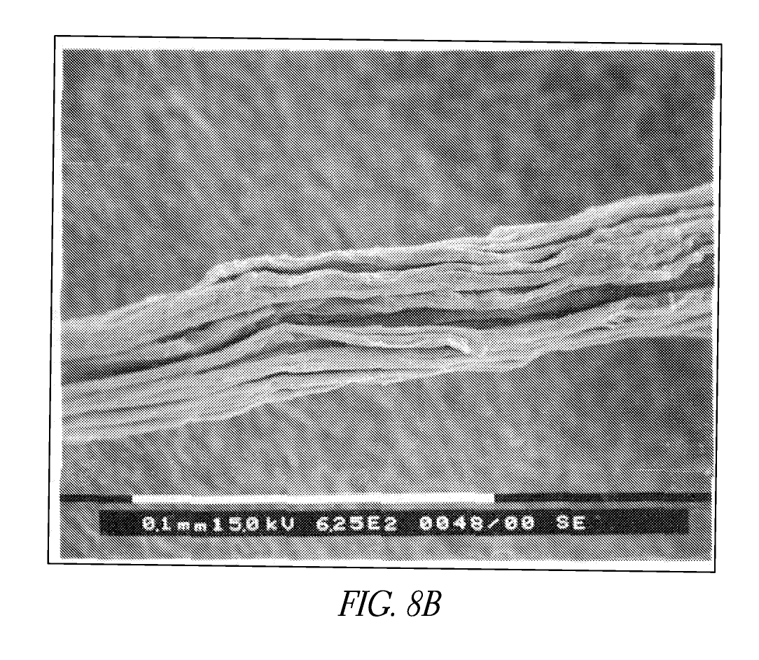

FIG. 8A-B illustrates SEM images of individual chitosan fibrid pulp obtained by the direct precipitation of sheared chitosan solutions. The fibrid pulp exhibit the increased surface area similar to that of chitosan fibrids described with reference to FIG. 7.

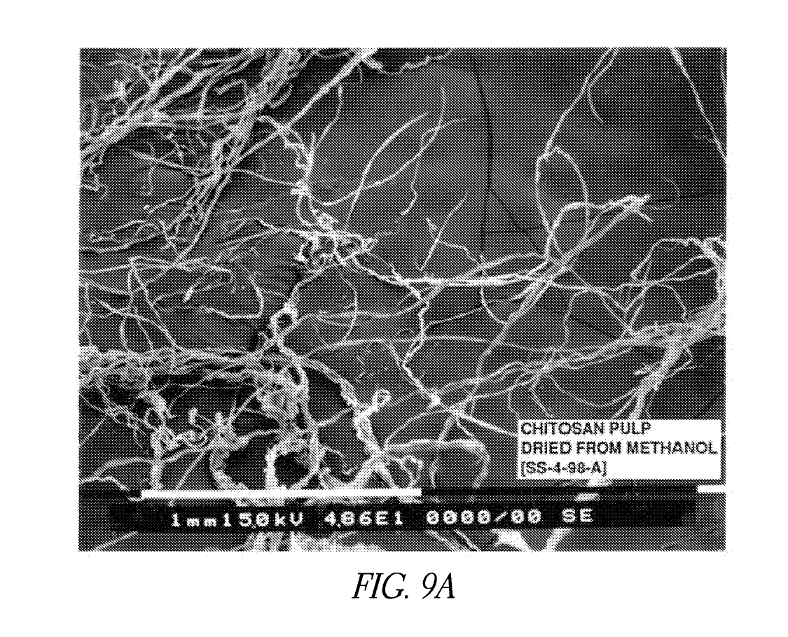

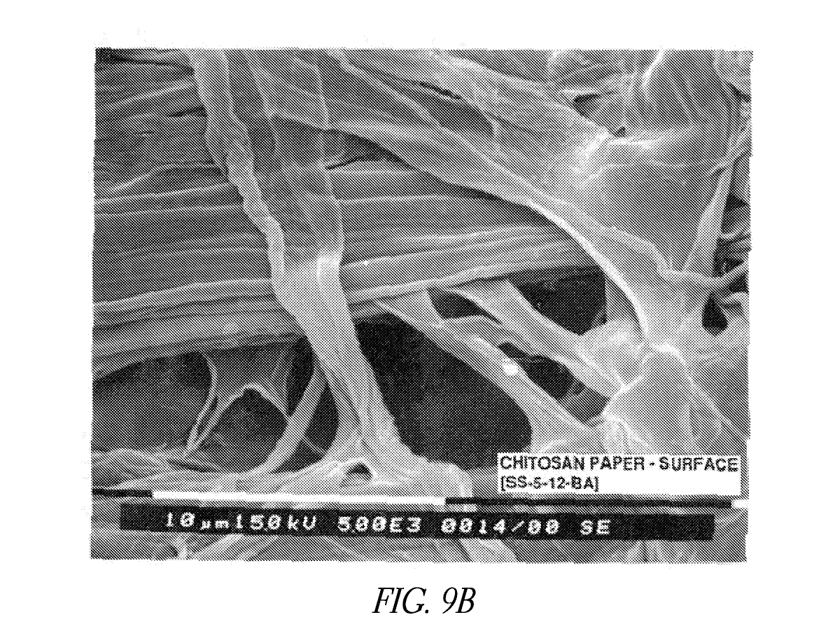

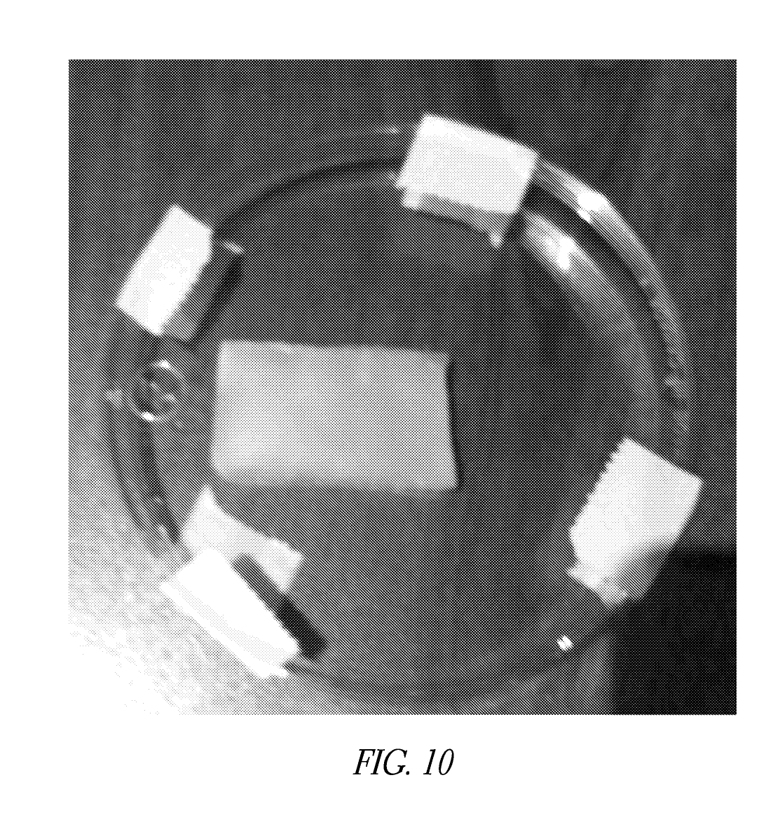

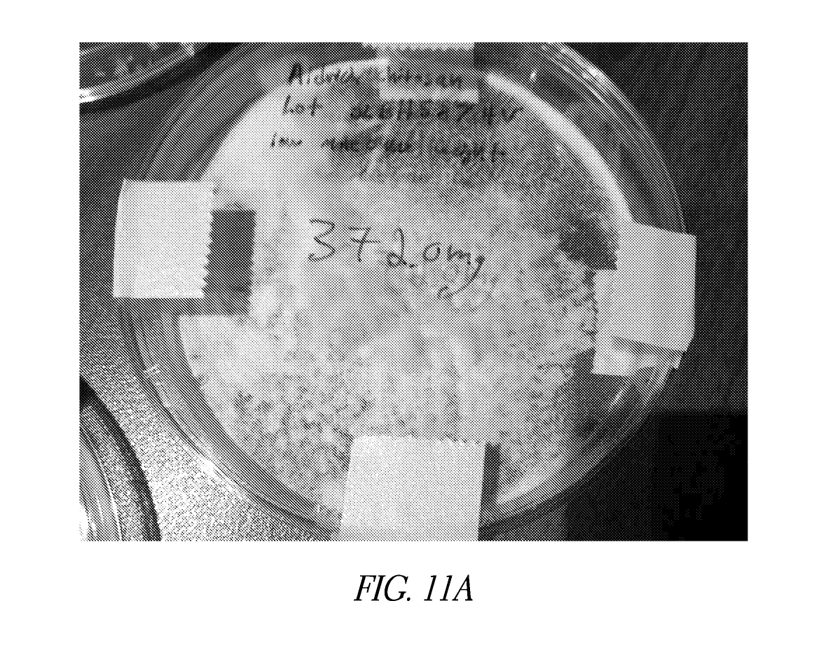

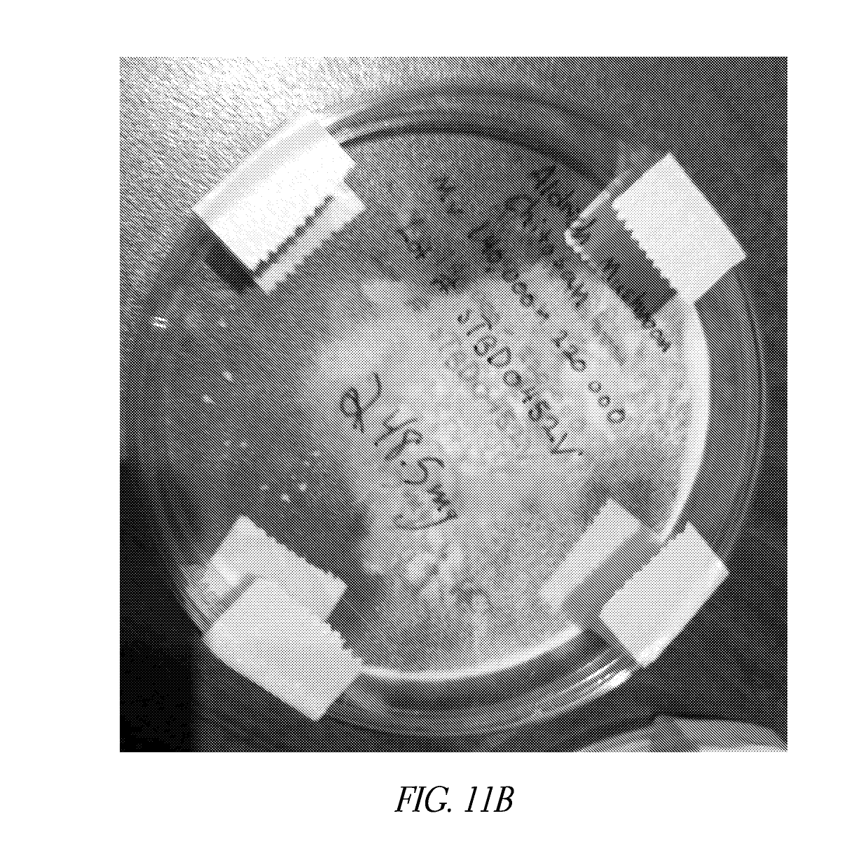

FIG. 9A-B illustrates SEM images of chitosan fibrids. FIG. 9A illustrates an SEM image of chitosan fibrids processed with methanol and obtained from shear precipitated chitosan solution. The scale bar represents 1 mm. The chitosan fibrids can be formed into films or paper-like structures. In some embodiments, the films or paper-like structures can be used for topical or internal medical applications as a hemostat and/or drug delivery vehicle. FIG. 9B shows film or paper-like structure obtained from chitosan fibrids. The scale bar represents 10 microns. In some embodiments, the films or paper-like structure can be arranged in a layered configuration to form a larger or thicker chitosan material. In some embodiments, the chitosan fibrid material can include a medicament or other agents that assist in wound healing and/or medicaments or agents that are to be delivered to the target implantation site.

The type of chitosan material formed from chitosan fibrids, shards, films, and/or ribbons will be determined by the intended use. Chitosan fibrids, shards, films, and/or ribbons can be formed into various materials such as a chitosan puff, fleece, film, or sheet similar to chitosan microfibers. In some embodiments, the chitosan fibrid, shards, films, and/or ribbons materials can have lower endotoxin levels than achieved utilizing chitosan microfibers because of the increased surface area exposed to the purification and treatment techniques of nitrogen plasma and gamma irradiation treatment as described herein.

Hydrogel with Chitosan

In some embodiments, the chitosan can be formed into a hydrogel material which can be used in similar applications as described with reference to the microfibril chitosan. A hydrogel is a gel in which the swelling agent is water or other liquid solution. Hydrogels can include a solid three-dimensional cross-linked network containing a dispersion of water molecules. Hydrogels can be inherently adhesive, and because of their significant water content, can possess a degree of flexibility very similar to natural tissues. Hydrogels can be formed of natural or synthetic polymers.

Chitosan can be used in hydrogels intended for internal surgical or medical uses. Chitosan's biocompatablity and mucoadhesive properties enable it to be used in a chitosan hydrogel material for an implantable device as described herein. In some embodiments, the chitosan material used in the hydrogel can be in the form of a chitosan powder, flake, fiber, and/or other chitosan materials known in the art. Chitosan materials for use in hydrogels possess the same purification restrictions as discussed herein with reference to chitosan fibers. The γ-irradiation in a nitrogen field, nitrogen-gas based plasma treatment, and/or other treatment methods to purify the chitosan can be utilized to ensure endotoxin removal suitable for the use of the chitosan for internal medical procedures or treatments. A depyrogenated chitosan hydrogel matrix can be produced. The chitosan hydrogel matrix can be implanted or injected into a target region. In some embodiments, the chitosan hydrogel matrix can include a medicament, therapeutic agent, or other agent.

### Chitosan Nanoparticles

Other forms of chitosan can be subjected to the chitosan purification treatment as described herein. Chitosan nanoparticles similar to those described in Applicants' copending International Patent Application No. PCT/US2013/30582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of this application is hereby incorporated by reference. Chitosan nanoparticles can have various applications as a drug delivery device and can be utilized to deliver various molecules to a targeted site. The biocompatablity of chitosan and the endotoxin removal techniques described herein allow chitosan nanoparticles to be effective targeted drug delivery devices that can be used in various applications.

The nanoparticles of various embodiments can have an average particle size of about 0.5, 0.6, 0.7, 0.8, 0.9, 1, 1.1, 1.2, 1.3, 1.4, 1.5, 1.6, 1.7, 1.8, 1.9, or 2 μm or more, e.g., 1 nm to 2000 nm or more. The size may depend on the drug to be encapsulated or the condition to be treated. In other embodiments, average particle size may be less than about 0.5 μm (500 nm), or 400, 300, 200, 100, 90, 80, 70, 60, 50, 40, 30, 20, 10, 9, 8, 7, 6, 5, 4, 3, 2, 1 nm or less. In various embodiments, the particles are of a substantially uniform size distribution, that is, a majority of the particles present have a diameter generally within about ±50% or less of the average diameter, within about ±45%, 40%, 35%, 30% or less of the average diameter, within ±25% or less of the average diameter, or within ±20% or less of the average diameter. The term “average” includes both the mean and the mode.

### Chitosan Powder

Additional forms of chitosan material can be used to enhance the ability to purify the chitosan material. Chitosan can be ground into smaller particles, granules, and/or powders. The powder structure of the chitosan, similar to chitosan shards, provides a sufficient surface area to volume ratio for improved depyrogenation and purification as described herein with reference to the chitosan shards or fibrids.

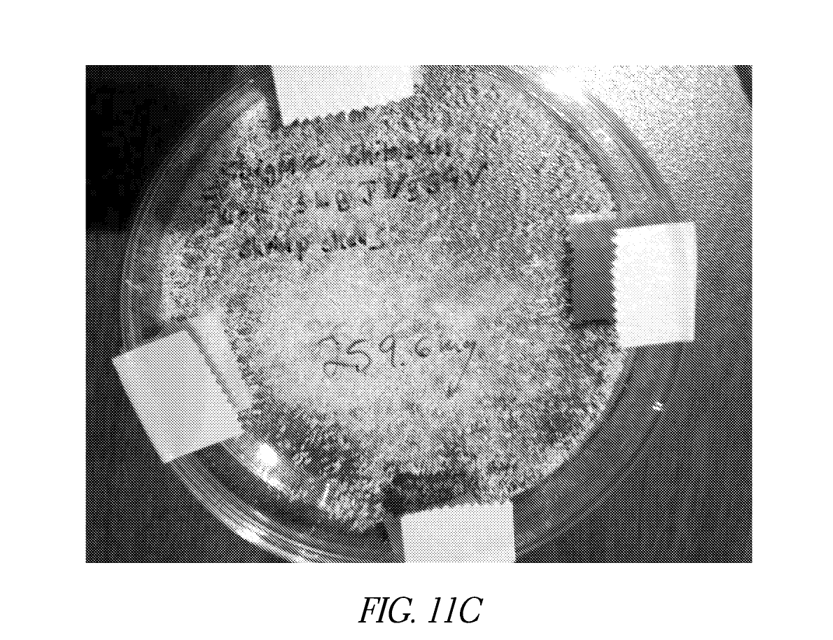

FIG. 10 is an embodiment of a chitosan pad. The chitosan pad as shown in FIG. 10 is an embodiment of a pad that can be treated with the purification techniques described herein. FIGS. 11A-C show pictures of various small particle chitosan materials. The chitosan small particles or powders can be treated with the plasma purification techniques described herein. FIG. 11A is a picture of a chitosan powder and/or small particles made from low molecular weight chitosan. FIG. 11B is a picture of chitosan powder and/or small particles derived from mushroom chitosan. FIG. 11C is a picture of a chitosan powder and/or small particles derived from shrimp shells. The chitosan powders of FIGS. 11A-C depict embodiments of chitosan powders or small particles that can be utilized during plasma treatment and provide a surface area to volume ratio that allows effective depyrogenation and purification of the chitosan material that exceeds the depyrogenation and purification that can be achieved with chitosan pads similar to that as depicted in FIG. 10.

The surface area of the chitosan pad exposed to the plasma treatment and/or other treatment techniques is limited in comparison to the entire volume of the chitosan pad. The limited surface area exposed to treatment allows for the interior region of the pad to be less affected or potentially not affected at all by the depyrogenation treatment. In contrast, the chitosan powders or small particles as depicted in FIGS. 11A-C are chitosan materials with a high exposed surface area. The exposed surface area of the powder can be effectively treated with the depyrogenation techniques as described herein. The unexposed surface area is much more limited than the chitosan pad and therefore, the almost the entire material can be depyrogenated. The treated chitosan powder can then be utilized topically or internally in powder form or formed into end products to be applied to the treated region.

An individual granule of the chitosan powder or small particle can be between less than or equal to 10 μm to greater than or equal to 250 μm in diameter. In some embodiments, the granule of chitosan powder or small particles can be about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, about 45 μm to about 50 μm, about 50 μm to about 55 μm, about 55 μm to about 60 μm, about 60 μm to about 65 μm, about 65 μm to about 70 μm, about 70 μm to about 75 μm, about 75 μm to about 80 μm, about 80 μm to about 85 μm, about 85 μm to about 90 μm, about 90 μm to about 95 μm, about 95 μm to about 100 μm, about 105 μm to about 110 μm, about 110 μm to about 115 μm, about 115 μm to about 120 μm, about 120 μm to about 125 μm, about 125 μm to about 130 μm, about 130 μm to about 135 μm, about 105 μm to about 110 μm, about 115 μm to about 120 μm, about 120 μm to about 125 μm, about 125 μm to about 130 μm, about 130 μm to about 135 μm, about 135 μm to about 140 μm, about 140 μm to about 145 μm, about 145 μm to about 150 μm, about 150 μm to about 155 μm, about 155 μm to about 160 μm, about 160 μm to about 165 μm, about 165 μm to about 170 μm, about 170 μm to about 175 μm, about 175 μm to about 180 μm, about 180 μm to about 185 μm, about 185 μm to about 190 μm, about 190 μm to about 195 μm, about 195 μm to about 200 μm, about 200 μm to about 205 μm, about 205 μm to about 210 μm, about 210 μm to about 215 μm, about 215 μm to about 220 μm, about 220 μm to about 225 μm, about 225 μm to about 230 μm, about 230 μm to about 235 μm, about 235 μm to about 240 μm, about 240 μm to about 245 μm, and about 245 μm to about 250 μm. In some embodiments, the chitosan powder or small particle can be on average about 28 μm in diameter.

While a uniform size distribution may be present, individual particles having diameters above or below the disclosed range may also be present, and may even constitute the majority of the particles present, provided that a substantial amount of particles having diameters in the range are present. In other embodiments, it may be desirable that the particles constitute a mixture of two or more particle size distributions, for example, a portion of the mixture may include a distribution on nanometer-sized particles and a portion of the mixture may include a distribution of micron-sized particles. The particles of various embodiments may have different forms. For example, a particle may constitute a single, integrated particle not adhered to or physically or chemically attached to another particle. Alternatively, a particle may constitute two or more agglomerated or clustered smaller particles that are held together by physical or chemical attractions or bonds to form a single larger particle. The particles can be in dry form, or in the form of a suspension in a liquid.

In some embodiments, chitosan in nanoparticulate form, e.g., solid form chitosan nanoparticles comprising therapeutic agents in solid form (e.g., as a tablet, capsule, or implant) or nanoparticles in liquid suspension or slurry (e.g., for oral administration, intravenous administration, or implantation by injection) can be provided. Chitosan nanoparticles can be made by spray-drying aqueous solutions or dispersions of chitosan and one or more pharmaceutically active components, optionally with a surface modifier to form a dry powder which consists of aggregated chitosan nanoparticles. An aqueous dispersion of chitosan, pharmaceutically-active agent and surface modifier, when spray dried, can form pharmaceutically-active agent embedded chitosan nanoparticles. In one embodiment, compositions are provided containing nanoparticles which have an effective average particle size of less than about 2000 nm, less than about 400 nm, less than about 300 nm, less than about 250 nm, less than about 100 nm, or less than about 50 nm, as measured by light-scattering methods. By “an effective average particle size of less than about 1000 nm” it is meant that at least 50% of the pharmaceutically-active agent particles have a weight average particle size of less than about 1000 nm when measured by light scattering techniques. In some embodiments, at least 70% of the pharmaceutically-active agent particles have an average particle size of less than about 1000 nm, at least 90% of the pharmaceutically-active agent particles have an average particle size of less than about 1000 nm, or at least about 95% of the particles have a weight average particle size of less than about 1000 nm.

Spray drying is a process used to obtain a powder containing chitosan nanoparticulate/pharmaceutically-active agent particles following particle size reduction of the pharmaceutically-active agent in a liquid medium. In general, spray-drying may be used when the liquid medium has a vapor pressure of less than about 1 atm at room temperature. A spray-dryer is a device which allows for liquid evaporation and pharmaceutically-active agent powder collection. A liquid sample, either a solution or suspension, is fed into a spray nozzle. The nozzle generates droplets of the sample within a range of about 20 to about 100 μm in diameter which are then transported by a carrier gas into a drying chamber. The carrier gas temperature is typically between about 80 and about 200° C. The droplets are subjected to rapid liquid evaporation, leaving behind dry particles which are collected in a special reservoir beneath a cyclone apparatus.

If a liquid sample consists of an aqueous dispersion of chitosan nanoparticles and surface modifier, the collected product will consist of spherical aggregates of the chitosan nanoparticulate/pharmaceutically-active agent particles. If the liquid sample consists of an aqueous dispersion of nanoparticles in which an inert diluent material was dissolved (such as lactose or mannitol), the collected product will consist of diluent (e.g., lactose or mannitol) particles which contain embedded chitosan nanoparticulate/pharmaceutically-active agent particles. The final size of the collected product can be controlled and depends on the concentration of chitosan nanoparticulate/pharmaceutically-active agent and/or diluent in the liquid sample, as well as the droplet size produced by the spray-dryer nozzle.

In some instances it may be desirable to add an inert carrier to the spray-dried material to improve the metering properties of the final product. This may especially be the case when the spray dried powder is very small (less than about 5 μm) or when the intended dose is extremely small, whereby dose metering becomes difficult. In general, such carrier particles (also known as bulking agents) are too large to be delivered to the lung and simply impact the mouth and throat and are swallowed. Such carriers typically consist of sugars such as lactose, mannitol, or trehalose. Other inert materials, including non-chitosan polysaccharides and cellulosics, may also be useful as carriers.

Sublimation can be employed to obtain a chitosan nanoparticulate/pharmaceutically-active agent composition. Sublimation avoids the high process temperatures associated with spray-drying. In addition, sublimation, also known as freeze-drying or lyophilization, can increase the shelf stability of pharmaceutically-active agent compounds, particularly for biological products. Sublimation involves freezing the product and subjecting the sample to strong vacuum conditions. This allows for the formed ice to be transformed directly from a solid state to a vapor state. Such a process is highly efficient and, therefore, provides greater yields than spray-drying. The resultant freeze-dried product contains pharmaceutically-active agent and modifier(s).

The compounds of various embodiments can be provided in the form of a spray-dried powder, either alone or combined with a freeze-dried nanoparticulate powder. Spray-dried or freeze-dried nanoparticulate powders can be mixed with liquid or solid excipients to provide unit dosage forms suitable for administration. Freeze dried powders of a desired particle size can be obtained by freeze drying aqueous dispersions of pharmaceutically-active agent and surface modifier, which additionally contain a dissolved diluent such as lactose or mannitol.

Milling of aqueous chitosan/pharmaceutically-active agent solutions to obtain chitosan nanoparticulates may be performed by dispersing pharmaceutically-active agent particles or dissolving a soluble pharmaceutically-active agent in a liquid dispersion medium comprising chitosan and applying mechanical means in the presence of grinding media to reduce the particle size of the pharmaceutically-active agent to the desired effective average particle size. The particles can be reduced in size in the presence of one or more surface modifiers. Alternatively, the particles can be contacted with one or more surface modifiers after attrition. Other compounds, such as a diluent, can be added to the pharmaceutically-active agent/surface modifier composition during the size reduction process. Dispersions can be manufactured continuously or in a batch mode.

Another method of forming a chitosan nanoparticle dispersion is by microprecipitation. This is a method of preparing stable dispersions of pharmaceutically-active agent and chitosan in the presence of one or more surface modifiers and one or more colloid stability enhancing surface active agents free of any trace toxic solvents or solubilized heavy metal impurities. Such a method comprises, for example, (1) dissolving chitosan and the pharmaceutically-active agent in a suitable solvent with mixing; (2) adding the formulation from step (1) with mixing to a solution comprising at least one surface modifier to form a clear solution; and (3) precipitating the formulation from step (2) with mixing using an appropriate nonsolvent. The method can be followed by removal of any formed salt, if present, by dialysis or diafiltration and concentration of the dispersion by conventional means.

In a non-aqueous, non-pressurized milling system, a non-aqueous liquid having a vapor pressure of about 1 atm or less at room temperature and in which the chitosan and pharmaceutically-active agent substance is essentially insoluble may be used as a wet milling medium to make a chitosan nanoparticulate/pharmaceutically-active agent composition. In such a process, a slurry of pharmaceutically-active agent and surface modifier may be milled in the non-aqueous medium to generate chitosan nanoparticulate/pharmaceutically-active agent particles. Examples of suitable non-aqueous media include ethanol, trichloromonofluoromethane, (CFC-11), and dichlorotetafluoroethane (CFC-114). An advantage of using CFC-11 is that it can be handled at only marginally cool room temperatures, whereas CFC-114 requires more controlled conditions to avoid evaporation. Upon completion of milling the liquid medium may be removed and recovered under vacuum or heating, resulting in a dry nanoparticulate composition.

In a non-aqueous, pressurized milling system, a non-aqueous liquid medium having a vapor pressure significantly greater than 1 atm at room temperature may be used in the milling process to make chitosan nanoparticulate/pharmaceutically-active agent compositions. The milling medium can be removed and recovered under vacuum or heating to yield a dry nanoparticulate composition.

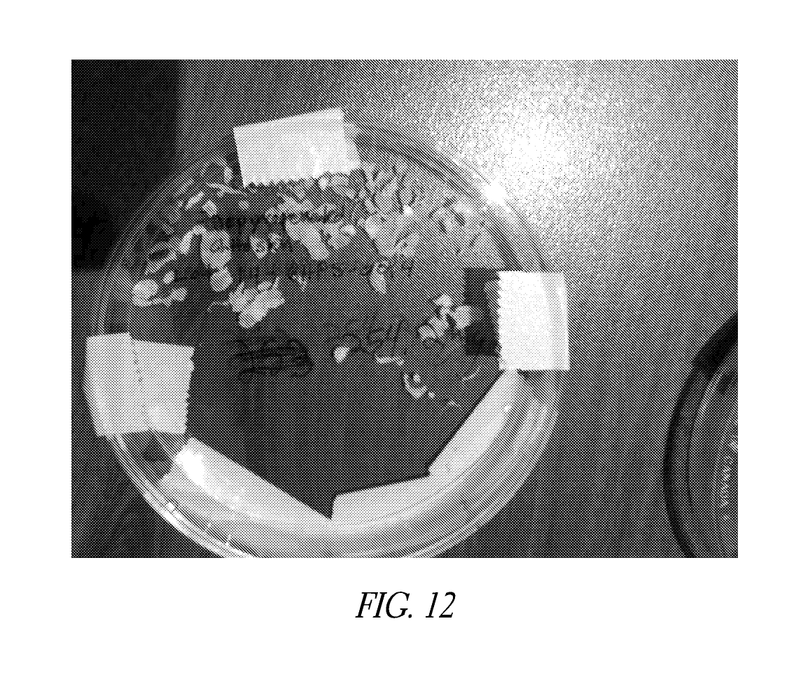

FIG. 12 is a picture of chitosan flakes that can be treated with depyrogenation techniques as described herein. The chitosan flakes, similar to the powder, also provide and increased surface area to volume ration that allows for the improved depyrogenation treatments. Additionally, in some embodiments, the chitosan flakes can be utilized as the starting product to create chitosan powder.

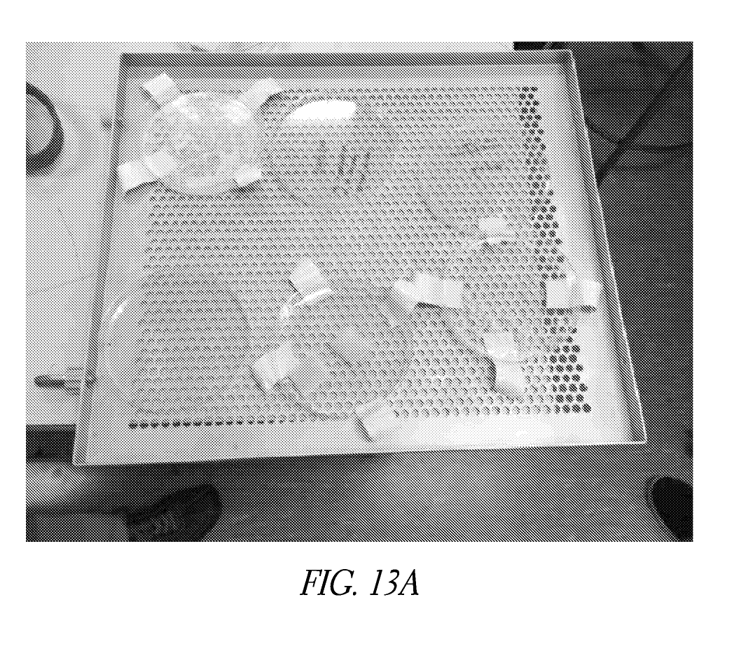

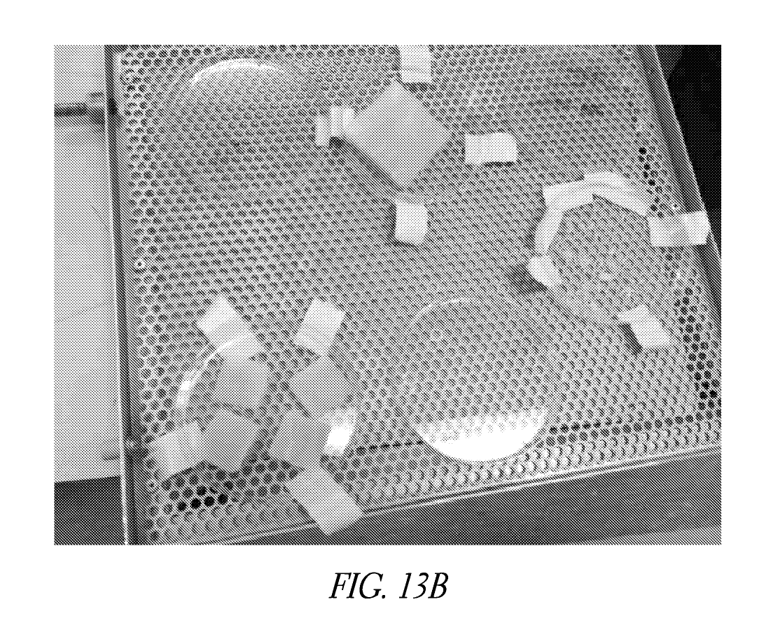

FIGS. 13A-B are pictures of chitosan powder, chitosan flakes, and chitosan pads. FIG. 13A shows embodiments of chitosan powder and a chitosan pad that are treated with plasma depyrogenation techniques described herein. FIG. 13B shows embodiments of chitosan flakes and chitosan pads of different sizes that are treated with plasma depyrogenation techniques described herein. FIGS. 13A-B show the relative sizes of the chitosan pad, flake, and powder material and thereby the available surface area for depyrogenation treatments.

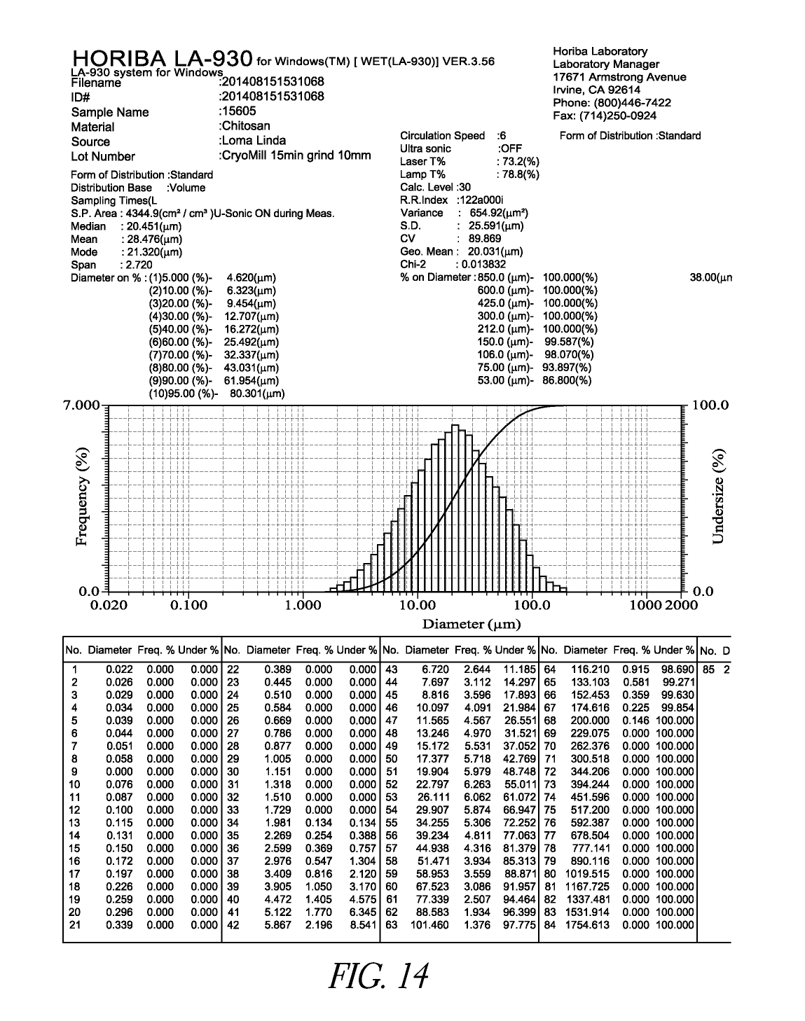

The powder or granules can vary in size. The dimensions of the powder, granules, or small chitosan particles must be sufficient to allow the particles to maintain their beneficial characteristics as described herein. Chitosan can be ground into chitosan powder utilizing grinding techniques known in the art. The chitosan powder can be derived from chitosan flakes, chitosan foam, chitosan shards, chitosan blocks, and/or other chitosan materials. The chitosan material can be cryomilled to produce the chitosan powder. Chitosan is a soft material as compared to cellulose or paper. The chitosan material can be frozen with liquid nitrogen and ground with zirconium grinding balls in order to micronize the material. By micronizing the surface area relative to volume is increased. The nitrogen plasma is a surface reactor or treatment that treats to a depth of 15-20 microns of the material. The material or fine powder can be treated with N2 plasma and depyrogenation and sterilization treatments as described herein. FIG. 14 shows the results from cryomilled chitosan material.

Chitosan flakes can be ground to make chitosan powder. The chitosan material can be treated with liquid nitrogen prior to milling or grinding to make the material soft enough to be milled. In some embodiments, the milling and/or grinding with low liquid N2 temperatures and zirconium ball pulverization can produce fine chitosan powder with a low endotoxin level even without plasma depyrogenation treatments of the material.

For example, chitosan flakes can be ground and/or cryomilled for about 15 to about 30 minutes using grinding balls. The grinding balls can be a hard material including, a metal such as steel or zirconium oxide. The grinding balls can be any size appropriate to achieve the desired size of the chitosan powder including, for example, grinding balls less than or equal to about 10 μm to greater than or equal to about 75 μm in diameter. In other examples, the grinding balls can be less than or equal to 5 mm in diameter or greater than or equal to 10 mm in diameter.

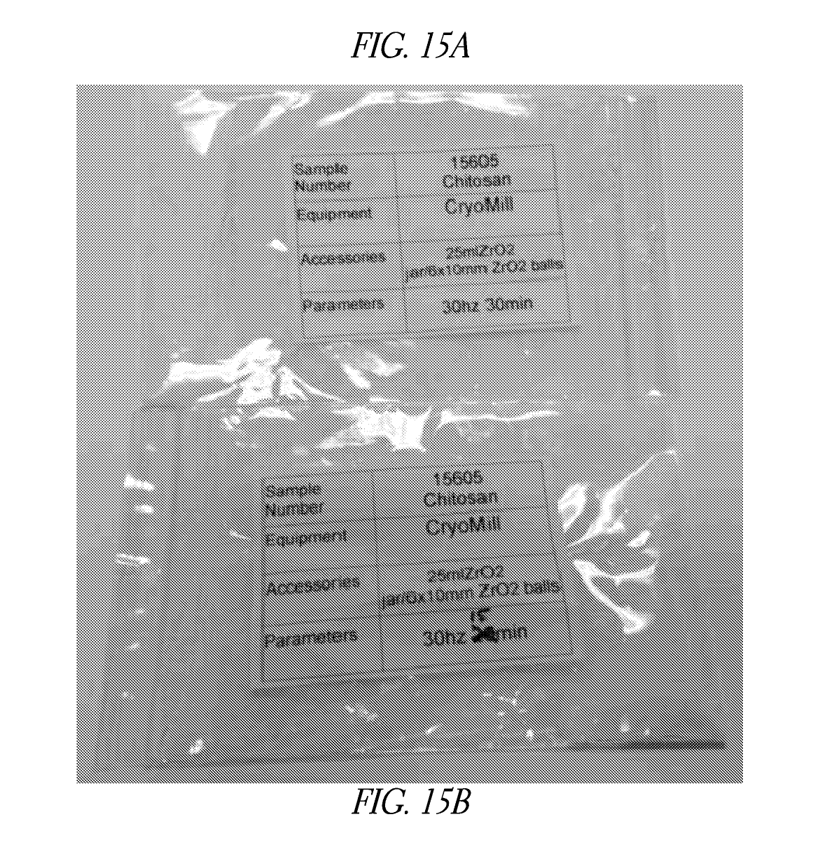

FIGS. 15A-B show chitosan powders that have been cryomilled. FIG. 15A shows chitosan powder which has been cryomilled using a 25 ml ZrO₂ jar with 6×10 mm ZrO₂ balls. The chitosan material was ground at 30 hz for 30 minutes.

FIG. 15B shows chitosan powder which has been cryomilled using a 25 ml ZrO₂ jar with 6×10 mm ZrO₂ balls. The chitosan material was ground at 30 hz for 15 minutes.

The chitosan material can be ground for less than or equal to 15 minutes to greater than or equal to 30 minutes. The chitosan material can be ground for 15 minutes to 20 minutes, 20 minutes to 25 minutes, and 25 minutes to 30 minutes or longer than 30 minutes. The chitosan material can be ground with various size grinding balls. The same size grinding balls or different size grinding balls can be used at one time. The balls can be any metal or hard material including, for example, steel or zirconium oxide grinding balls. The grinding balls can be less than or equal to about 10 μm to greater than or equal to about 80 μm in diameter. The grinding balls can be between about 10 μm, about 20 μm to about 30 μm, about 30 μm to about 40 μm, about 40 μm to about 50 μm, about 50 μm to about 60 μm, about 60 μm to about 70 μm, and about 70 μm to about 80 μm in diameter.

In some examples, the micronized chitosan can be produced by cryo-ball milling (using different ball sizes and times) or by cryo-jet milling. Below is an analysis of the data conducted to analyze the size distributions of chitosan microparticles after milling.

Chitosan polyprolate (CS) derived from arctic crab shells in the form of 1-10 mm flakes was obtained. The CS flakes were filtered to remove flakes>8 mm. Approximately 33 g of filtered CS was placed in a 25 mL zirconium oxide jar. Six milling balls made of either zirconium oxide (10 mm diameter) or stainless steel (5 mm) were added to the jar and the CS flakes were subsequently milled under cooling with liquid nitrogen at 30 Hz for up to 30 minutes in the Retsch CryoMill system (Verder Scientific, Inc., Newtown, Pa.). For cryo-jet milling, chitosan flakes were filtered to remove flakes>8 mm. 470 g of filtered CS was placed in the Micron-Master jet pulverization system (The Jet Pulverization Co., Inc., Moorestown, N.J.), cooled with liquid nitrogen, and then milled with a jet stream of liquid nitrogen for 30 minutes. Particle size analysis was performed by suspending chitosan in water and measuring particle size with a Horiba LA-930 laser diffraction analyzer (HORIBA Instruments, Inc., Irvine, Calif.). Below is a size distribution of chitosan microparticles after milling. In some examples, the cryo-milling has no effect on the pyrogen levels of the chitosan.

The table below shows the resulting size distributions of chitosan microparticles after milling. The mean, median, and D₉₅ is provided. The D₉₅ represents the diameter at which 95% of the sample's mass is composed of particles with a diameter smaller than the D₉₅ value. The D₉₅ diameter is therefore larger than 95% of the particles in the sample distribution.

TABLE 3

| | Mean | | Median | | D95 | |
|---|---|---|---|---|---|---|
| Cryo-ball Mill (10 mm balls; | 84.5 | um | 71.0 | um | 209.7 | um |
| 15 mins) | | | | | | |
| Cryo-ball Mill (10 mm balls; | 66.8 | um | 52.2 | um | 179.0 | um |
| 30 mins) | | | | | | |
| Cryo-ball Mill (5 mm balls; | 28.5 | um | 20.4 | um | 80.3 | um |
| 30 mins) | | | | | | |
| Cryo-jet Mill (30 mins) | 16.05 | um | 15.62 | um | 24.24 | um |

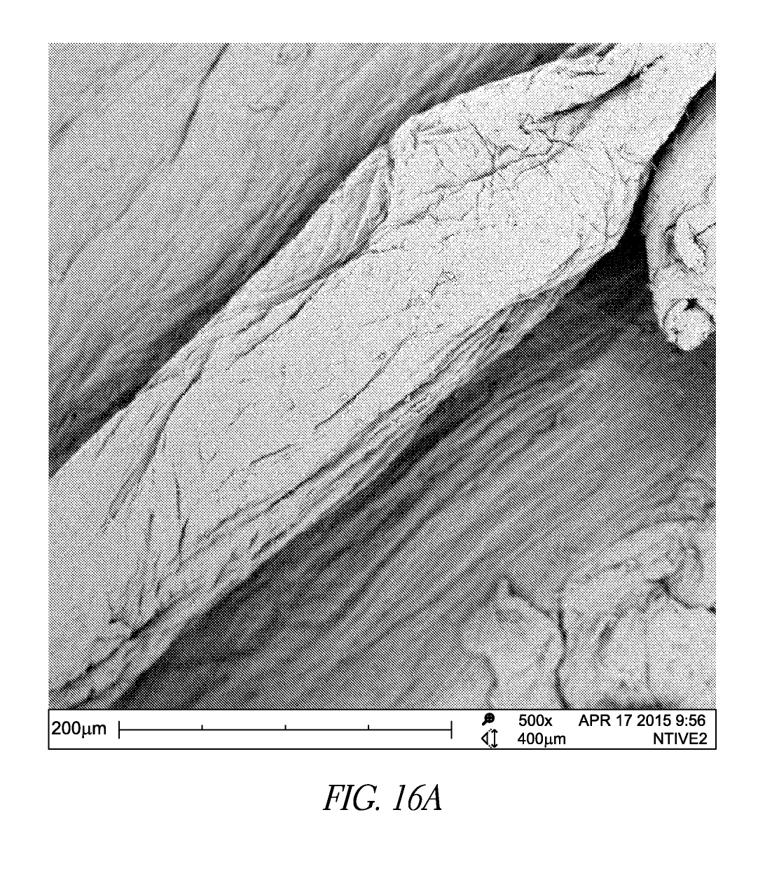

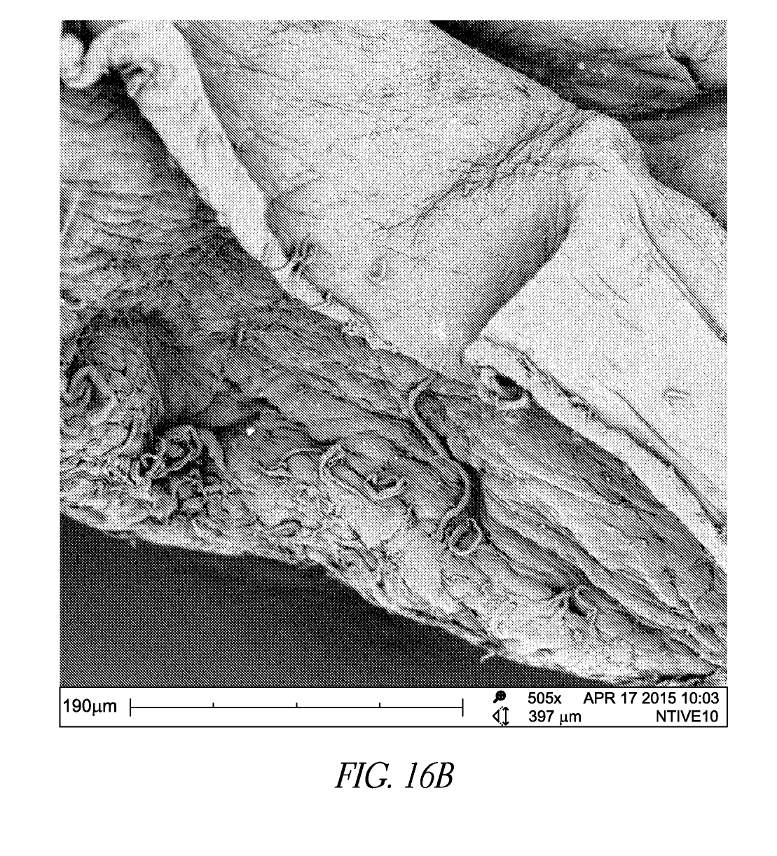

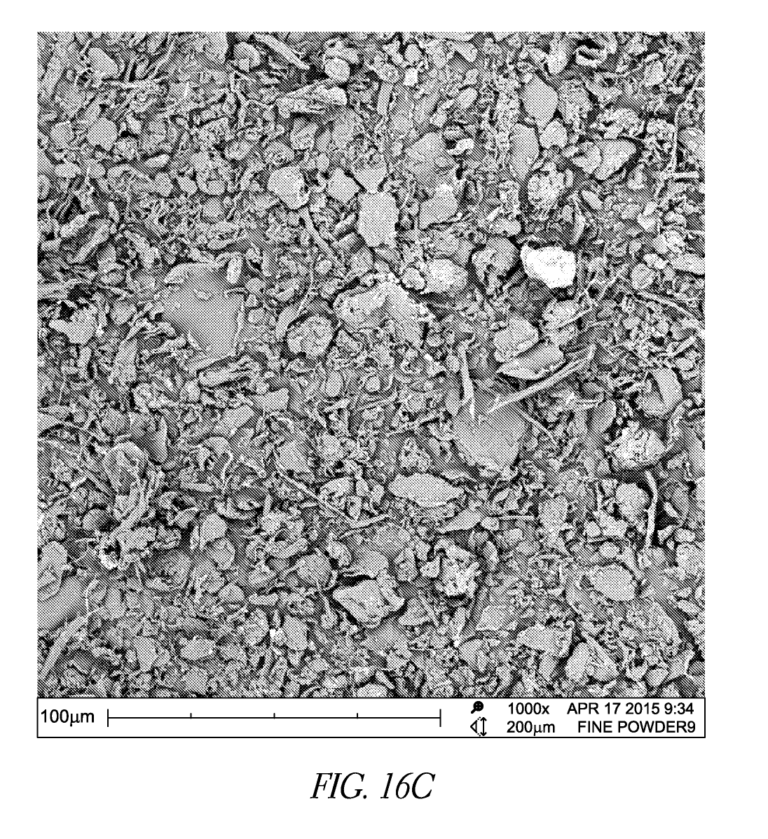

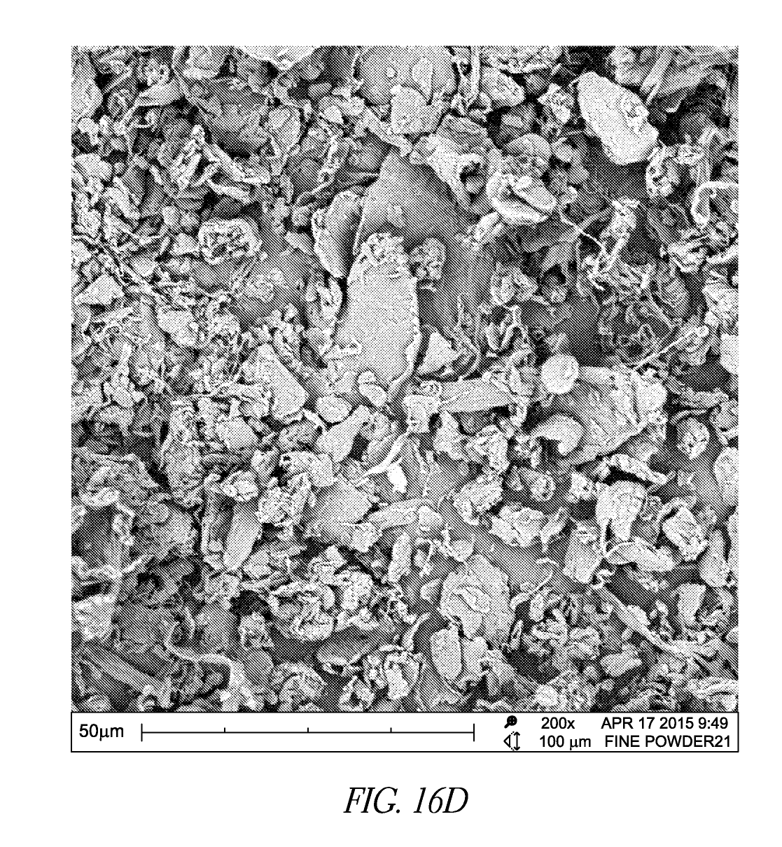

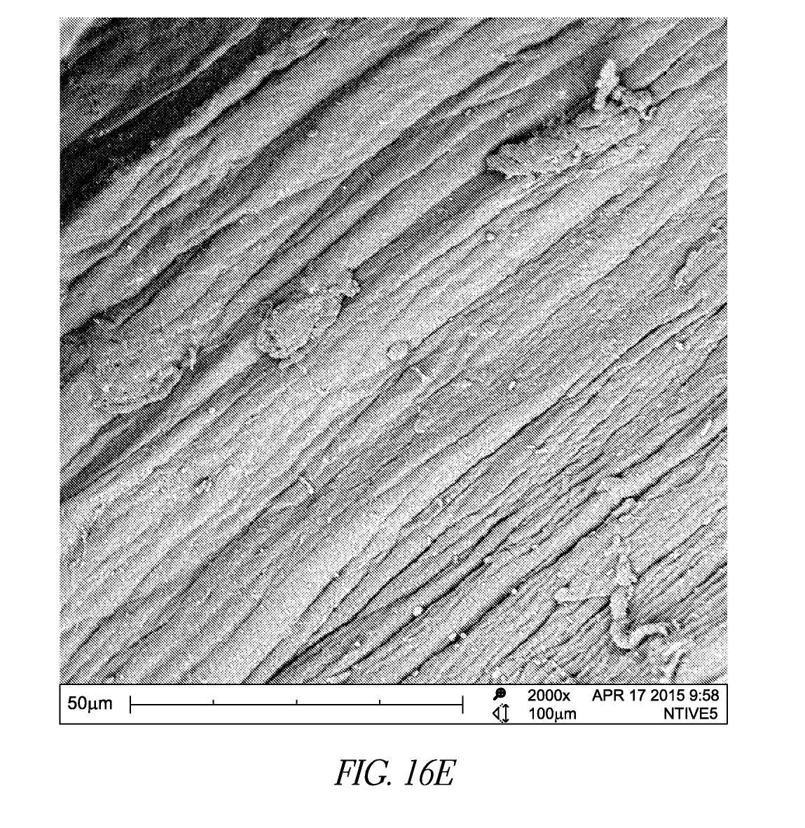

FIGS. 16A-E illustrate scanning electron microscopic images of chitosan flakes before and after milling. FIG. 16A-B illustrate chitosan flakes at 500 times magnification before milling. FIG. 16C illustrates a cryo-jet milled chitosan at 1000 times magnification. FIG. 16D illustrates a 2000 times magnification of a cryo jet milled CS. FIG. 16E illustrates a 2000 times magnification of a chitosan flake.

Cryomilling is a variation of mechanical milling, in which powders or other solids are milled in a cryogen (usually liquid nitrogen, liquid carbon dioxide, or liquid argon) slurry or at a cryogenics temperature under processing parameters, so a nanostructured microstructure is attained. Cryomilling takes advantage of both the cryogenic temperatures and conventional mechanical milling. The extremely low milling temperature suppresses recovery and recrystallization and leads to finer grain structures and more rapid grain refinement. The embrittlement of the sample makes even elastic and soft samples grindable. Tolerances less than 5 μm can be achieved. The ground material can be analyzed by a laboratory analyzer. Freezer milling is a type of cryogenic milling that uses a solenoid to mill samples. The solenoid moves the grinding media back and forth inside a container, grinding the sample down to a desired degree of fineness. The idea behind using a solenoid is that the only moving part in the system is the grinding media inside the vial.

The ground chitosan powder can be treated with plasma depyrogenation treatments as described herein. In other embodiments, the chitosan material, for example the chitosan flake, can be treated with the plasma depyrogenation treatments described herein prior to grinding of the chitosan material into the chitosan powder. The chitosan powder endotoxin contamination can be reduced to less than 20 EU/device or less than 0.5 EU/gram. The increased surface area to volume ratio of the chitosan powder allows for a greater reduction in endotoxin contamination of the material. Additionally, the tumbling or rotating of the powder within the chamber during plasma treatment can allow for a more even treatment which can result in a lower endotoxin level than chitosan that maintains stationary during plasma treatment.

The depyrogenated chitosan powder can be utilized to create a chitosan hemostatic product and/or chitosan drug delivery product as described herein. For example, the chitosan powder can be used to form a chitosan pad, foam, sponge, gel, solution, or other chitosan product known in the art.

For example, depyrogenated chitosan powders and/or small particles can be disposed in a viscous solution or gel and delivered through an endoscopic port to a surgical site and used or implanted internally to control bleeding, as a therapeutic agent, and/or as a drug delivery vehicle. In some embodiments, the chitosan powder can be created by utilizing large chitosan flakes directly obtained from shellfish with multi centimeter dimensions.

The chitosan powder can be solubilized to form a chitosan solution. The chitosan can be combined with an acid to form a viscous solution or gel, similar in form and structure to a hydrogel. The solution can contain 1% chitosan and 2% acetic acid solution. Chitosan powder can be dissolved in acetic acid, lactic acid, glutamic acid, formic acid, or another acid of the like. Dissolving the chitosan in lactic acid can have increased biocompatibility. Organic or inorganic acids can be employed. In some examples, the acid is an organic acid.

In some embodiments, medicament, therapeutic agent, other agent, and/or other drugs can be added to the chitosan solution and delivered to the surgical site. Depyrogenated chitosan powder solution can be used as an excipient for a variety of drugs. For example, therapeutic agents can include cytokine interleukin-12 (IL-12). IL-12 can have a significant anti-tumor and anti-metastatic effect. The IL-12 treatment is a cancer immunotherapy and can generate cancer immunity. IL-12 augments natural killer (NK)/lymphocyte-activated killer cell activity, enhances cytolytic T cell generation, and induces interferon gamma (TNF-γ) secretion. IL-12 may provide significant protection against tumor re-challenge by potentiating immunologic memory and regulating T cell activity via proliferation of both activated CD4+ and CD8+ T cell subsets.

Other therapeutic agents including agents with anti-tumor and anti-metastatic effect can be used, for example, a chemotherapeutic agent. The chitosan powder and/or solution can include a medicament or other agents that assist in wound healing and/or medicaments or agents that are to be delivered to the target implantation site. In some embodiments, the therapeutic agent can include a medicament, an anti-infective agent, an antimicrobial, polyhexamethylene biguanide (hereinafter, “PHMB”), antibiotics, analgesics, healing factors, vitamins, growth factors, and nutrients and/or other agent known in the art.

Chitosan is a recognized drug delivery vehicle; however, it has not been utilized as a drug delivery vehicle clinically due to the high endotoxin levels of currently available materials. Additionally, IL-12 is a known immunotherapeutic agent; it has not been successfully applied clinically due to toxicity when given systemically. Chitosan is only used clinically as a topical hemostat because the material cannot be adequately depyrogenated for internal use with standard depyrogenation methods like ethylene oxide, γ-irradiation, heat, and/or electron beam without altering its advantageous functional properties. Thus, using the nitrogen plasma method as described herein to produce implantable, depyrogenated chitosan with unaltered functionality can enable chitosan and IL-12 or other medicament to be used as a treatment for cancer and/or other disease and chitosan to be used in many other ways clinically.

Several physical forms of a chitosan material are available for depyrogenation and endoscopic deployment. In some embodiments, it is necessary to identify an optimal chitosan form compatible with implantation via a laparoscopic port. For example, if the optimal form is a viscous chitosan solution or gel that can be injected or deployed to a surgical site or target site through an endoscope.

For example, in some embodiments, the chitosan solution can be produced by the production of fine powders from chitosan flakes or other material as described herein. The manufacture of chitosan powder from a chitosan flake is a method for producing a chitosan powder and ultimately a chitosan solution that may be more readily depyrogenated due to the increase in exposed surface area of the powder material. Chitosan powder formulation produced through this method can form chitosan material with an increased surface area than that of other chitosan materials. In some embodiments, the increased surface area can produce a chitosan material that can be potentially more vulnerable to nitrogen depyrogenation and resorption. In some embodiments, the chitosan powder formation can be amenable to laparoscopic implantation for internal medical purposes. In some embodiments, the chitosan powder formation formed by the cryomilled manufacturing process can have the necessary flexibility and rigidity for internal medical applications.

In some embodiments, a depyrogenated chitosan hydrogel matrix can be produced as described above and the chitosan hydrogel matrix can be implanted or injected into a target region with a therapeutic agent. In some embodiments, the chitosan can be depyrogenated using γ-irradiation in a nitrogen field, nitrogen-gas based plasma treatment, and/or another treatment methods to purify the chitosan. The depyrogenated chitosan can be formed into a hydrogel. Therapeutic agents that can be incorporated or mixed with the chitosan hydrogel can include any medicaments including, but not limited to, cytokine interleukin-12 (IL-12). IL-12 can be injected into, combined within, and/or seeded on the chitosan hydrogel. The chitosan hydrogel matrix can be implanted or injected into a target region with the therapeutic agent cytokine interleukin-12 (IL-12). The IL-12 can have significant anti-tumor and anti-metastatic effects in the target region as described in detail in Applicant's International Patent Application No. PCT/US2014/050188, filed Aug. 7, 2014, and directed to “SYSTEMS AND METHODS FOR THE TREATMENT OF BLADDER CANCER,” now published as WO 2015/021303. The entirety of this application is hereby incorporated by reference.

Additionally, the chitosan end product can contain medicinal agents and/or other active agents to assist in wound healing or drug delivery as described in detail herein. In some embodiments, the chitosan powder or flake can be constructed as a gel, foam, solution, puff, fleece, fabric, or sheet form. For example, in some embodiments, a depyrogenated chitosan nanoparticle can be produced as described herein and in Applicants' copending International Patent Application No. PCT/US2013/30582, filed Mar. 12, 2013, and directed to a “SUBSTANCES AND METHODS FOR THE TREATMENT OF CEREBRAL AMYLOID ANGIOPATHY RELATED CONDITIONS OR DISEASES”, now published as WO 2013/138368. The entirety of this application is hereby incorporated by reference. The chitosan nanoparticle can be depyrogenated using γ-irradiation in a nitrogen field, nitrogen-gas based plasma treatment, and/or another treatment methods to purify the chitosan.

In some embodiments, the depyrogenated chitosan nanoparticle can include a targeting agent that allows for targeted delivery of the chitosan nanoparticle to the treatment site. Additionally, in some embodiments, a depyrogenated chitosan nanoparticle can be implanted or injected into a target region. The depyrogenated chitosan nanoparticle can be delivered to the target region with a medicament, therapeutic agent, or other agent. For example, depyrogenated chitosan nanoparticle can be delivered to the target region with a therapeutic agent. The therapeutic agents can be incorporated into or encapsulated within the chitosan nanoparticle. The therapeutic agent can include cytokine interleukin-12 (IL-12) and/or other medicament or therapeutic agent. The chitosan nanoparticle can be implanted or injected into a target region with the therapeutic agent.

In some embodiments, the fine powder dissolved in solution can be utilized as an end product for internal and/or external purposes to promote wound healing and/or to act as a vehicle for drug delivery. The chitosan powder can be coupled to a drug or medicament as described herein that can act on the target tissue or target site. The chitosan powder can be coupled to medicinal agents through complementary binding techniques including, for example, streptavidin/biotin standard conjugation. In other embodiments, the chitosan powder can be coupled to medicinal agents through activation of the surface characteristics of the chitosan powder material. The activation of the surface can open up chitosan for binding and allow a medicinal agent or drug to be coupled to the chitosan powder.

Embodiments of a chitosan-based hemostatic textile can be provided in many forms depending upon the nature of the wound and the treatment method employed. For example, a puff, fleece, or sponge form can be for controlling active bleeding from an artery or vein, or for internal bleeding during laparoscopic procedures. In neurosurgery, where oozing brain wounds are commonly encountered, a flexible sheet formed of the hemostatic material can be used. Likewise, in oncological surgery, especially of the liver, a sheet form or sponge form of the hemostatic material can be employed, which is placed in or on the tumor bed to control oozing. In dermatological applications, a sheet form can be used. In closing punctures in a blood vessel, a puff form can be used. A suture form, such as a microsuture or a macrosuture, can be used in certain applications. In performing a laparoscopic partial nephrectomy a chitosan gel, solution, foam, sponge, shard, fibrid, and/or films can be applied to the surgical site.

In some embodiments, the network of chitosan powder and/or flakes need not be processed into a specific textile or foam, but instead can be packed, clumped, wadded together into a network of ribbons that resembles Easter basket grass, mixed, and/or suspended in a solution that can be applied to the surgical site. The solution can be a viscous solution with a viscosity sufficient enough to allow the chitosan material to be maintained in solution and the solution to remain localized to the surgical site. In some embodiments, the viscous solution is similar in structure and form to hydrogel materials as known in the art. The viscous solution allows the chitosan material to be applied to a specific surgical site and substantially maintain its position at the surgical site. In some embodiments, the viscosity allows the chitosan material solution to take on a regular shape and form similar to that of thin films or pads. These thin films or pads can be a gel or hydrogel material which a defined shape and flexibility. In contrast, in other embodiments, the viscous chitosan solution may not take on a defined structure or form. The viscosity of the chitosan material solution allows the solution to conform to the site to which it is applied and to remain localized to that site, however, the chitosan material solution can be an amorphous material which may not have a defined shape prior to application to the site. The chitosan material solution may be a strong and durable material that is flexible and malleable but retains continuity so that it can be moved as a unit and doesn't break apart when manipulated during implantation and use. In certain embodiments, a viscosity of the viscous chitosan solution is from 50 cps (at 20° C.) or less to 250,000 cps (at 20° C.) or more, in some embodiments from about 100 cps (at 20° C.) to 100,000 cps (at 20° C.), in some embodiments from about 1,000 cps (at 20° C.) to 70,000 cps (at 20° C.), and in some embodiments from about 5,000 cps (at 20° C.) to 50,000 cps (at 20° C.).

In some embodiments, embodiments of chitosan are amenable to all of these applications and configurations, and embodiments are envisioned in which devices made from such chitosan are formed and shaped accordingly.

In some embodiments, the production of chitosan materials and associated processing can use chitosan of relatively high molecular weight. Such high molecular weight chitosan can be amenable to formation into a final chitosan product that can be formed into a strong and durable material that is flexible and malleable but retains continuity so that it can be moved as a unit and doesn't break apart when manipulated during use.

While various embodiments of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not ‘limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of making a material, comprising:
processing chitosan into a powder chitosan material by a physical process;
irradiating the powder chitosan material under nitrogen plasma; and
combining the powder chitosan material with an acid to create a viscous solution.

2. The method of claim 1, further comprising utilizing chitosan flakes directly obtained from shellfish, the chitosan flakes having an average largest dimension of 1 centimeter or more.

3. The method of claim 2, further comprising grinding the chitosan flakes with zirconium grinding balls.

4. The method of claim 1, wherein the physical process comprises grinding of the chitosan to form the powder chitosan material.

5. The method of claim 1, wherein an individual granule of the powder chitosan material is 10 μm to 100 μm in diameter.

6. The method of claim 1, wherein an average diameter of individual granules of the powder chitosan material is from 10 μm to 100 μm in diameter.

7. The method of claim 1, wherein the acid is selected from the group consisting of acetic acid, lactic acid, glutamic acid, and formic acid.

8. The method of claim 1, wherein the acid is lactic acid.

9. The method of claim 1, further comprising processing the viscous solution into a hemostatic device comprising a network of the powder chitosan material.

10. The method of claim 1, further comprising processing the viscous solution into a drug delivery device comprising a network of the powder chitosan material and a drug.

11. The method of claim 1, additionally comprising treating the powder chitosan material under a nitrogen plasma.

12. The method of claim 11, additionally comprising soaking the powder chitosan material in an alcohol prior to treating with γ-irradiation or plasma.

13. The method of claim 11, additionally comprising treating the powder chitosan material under the nitrogen plasma for 30 minutes or more.

14. A method of making a material, comprising:
processing chitosan into a powder chitosan material by a physical process; and
irradiating the powder chitosan material under a nitrogen plasma while rotating the powder chitosan material during nitrogen plasma irradiation.

15. The method of claim 14, wherein the rotating is conducted by a tumbler plasma chamber.

16. The method of claim 14, further comprising utilizing chitosan flakes directly obtained from shellfish, the chitosan flakes having an average largest dimension of 1 centimeter or more.

17. The method of claim 16, further comprising grinding the chitosan flakes with zirconium grinding balls.

18. The method of claim 14, wherein the physical process comprises grinding of the chitosan to form the powder chitosan material.

19. The method of claim 14, wherein an individual granule of the powder chitosan material is 10 μm to 100 μm in diameter.

20. The method of claim 14, wherein an average diameter of individual granules of the powder chitosan material is from 10 μm to 100 μm in diameter.

21. The method of claim 14, additionally comprising treating the powder chitosan material under a nitrogen plasma.

22. The method of claim 21, additionally comprising soaking the powder chitosan material in an alcohol prior to treating with γ-irradiation or plasma.

23. The method of claim 21, additionally comprising treating the powder chitosan material under the nitrogen plasma for 30 minutes.

24. An apparatus comprising:
a drug delivery device, the device comprising a powder material, the powder material comprising chitosan, wherein the powder material is γ-irradiated under nitrogen.

25. The apparatus of claim 24, wherein the powder material is configured to be processed into a hemostatic device comprising a network of powder material dissolved in an acid.

26. The apparatus of claim 24, wherein the powder material is configured to be processed into a drug delivery device comprising a network of the powder material dissolved in an acid.

27. The apparatus of claim 24, wherein the powder material is formed from a physical process comprises grinding of chitosan flakes to form the powder material.

28. The apparatus of claim 24, wherein an individual granule of the powder material is 10 μm to 100 μm in diameter.

29. The apparatus of claim 24, wherein the powder material is treated under a nitrogen plasma.

30. The apparatus of claim 24, the powder material is soaked in an alcohol prior to treating with γ-irradiation or plasma.

31. The apparatus of claim 24, the powder material is treated under a nitrogen plasma for 30 minutes or more.

32. The apparatus of claim 24, containing <20 endotoxin units.

33. The apparatus of claim 24, containing <20 endotoxin units per gram of chitosan.

34. The apparatus of claim 24, containing <20 endotoxin units per gram of chitosan.
